(19) United States
(12) Patent Application Publication
(10) Pub. No.: US 2009/0221095 A1
(43) Pub. Date: Sep. 3, 2009

(54) Colorimetric Screening of DNA Binding/Intercalating Agents with Gold Nanoparticle Probes

(75) Inventors: Chad A. Mirkin, Wilmette, IL (US); Min Su Han, Kyung (KR); Abigail K.R. Lytton-Jean, Cambride, MA (US)

Correspondence Address: MARSHALL, GERSTEIN & BORUN LLP, 233 SOUTH WACKER DRIVE, 6300 SEARS TOWER, CHICAGO, IL, 60606-6357, US

(73) Assignee: NORTHWESTERN UNIVERSITY, EVANSTON, IL (US)

(21) Appl. No.: 12/090,065

(22) PCT Filed: Oct. 13, 2006
(86) PCT No.: PCT/US06/40124

§ 371 Date: Dec. 4, 2008

Related U.S. Application Data
(60) Provisional application No. 60/726,640, filed on Oct. 13, 2005.
(60) Provisional application No. 60/749,745, filed on Dec. 13, 2005.
(60) Provisional application No. 60/839,979, filed on Aug. 24, 2006.

(51) Int. Cl. G01N 33/566

(52) U.S. Cl. 436/501

(57) ABSTRACT

Methods are provided to identify and characterize compounds that bind with duplex and triplex polynucleotides.

### GRANT FUNDING DISCLOSURE

The work described herein was supported by grants from the NIH (1DP1OD000285-1), DURINT/AFOSR F49620-01-1-0401, the NSF/NSEC (EEC-0118025/003) and CCNE 1U54CA119341. Accordingly, the US government may have certain rights to the invention.

### FIELD OF THE INVENTION

The field of the invention relates to methods to identify compounds that interact with duplex or triplex polynucleotides complexes.

### BACKGROUND

Combinatorial chemistry is a powerful tool which enables scientists to synthesize many compounds within a short time period. This ability to synthesize large libraries of compounds has enabled the development of many potential anticancer drugs. However, one of the bottlenecks in drug discovery is the selection of drug candidates from the many compounds within these libraries. To overcome this problem, high-throughput screening methods are used to screen large libraries of potential drug candidates for biological activity. For example, many anticancer drugs such as doxorubicine, daunorubicin and amsacrine, are known to reversibly interact with DNA to form a drug/DNA complex. Generally, the strength of binding between the anticancer drugs and DNA correlates with the drug's biological activity and, therefore, is important in the screening process.

Triplex DNA binders, in addition to duplex DNA binders, represent another potential strategy for the treatment of genetic-based diseases. A promising approach involves the use of triplex forming oligonucleotides (TFOs). Triple helix nucleic acids, or triplex structures, are formed through sequence specific Hoogsteen, or reverse Hoogsteen, hydrogen bond formation between a single-stranded TFO and purine bases in the major groove of a target duplex. Because TFOs can achieve sequence-specific recognition of genomic DNA, they can, in principle, be used to modulate gene expression by interfering with transcription factors that bind to DNA. However, at present only purine-rich sequences can be targeted and the resultant triplex structure is less stable than the analogous duplex. This inherent instability has prompted research efforts to develop molecules that selectively bind to such triplex structures to stabilize the TFO-duplex complex. Potentially, triplex specific binding molecules could be used in conjunction with TFOs to achieve control of gene expression by interfering with transcription factors that bind to DNA. Molecules identified as triplex binders include benzoindoloquinoline, benzopyridoquinoxaline, naphthyquinoline, acridine, and anthraquinone derivatives.

In the past, typical screening processes for compounds with the ability to bind duplex polynucleotides or triplex have included competitive dialysis, mass spectroscopy, electrophoresis, ultraviolet (UV)/visible electromagnetic melting experiments, nuclear magnetic resonance, light scattering, and electrochemistry, none of which are applicable to high-throughput screening processes. However, with the development of combinatorial libraries which can produce large numbers of potential drug candidates, high-throughput screening strategies have become a necessary part of drug development and only recently have high-throughput compatible fluorescence screening protocols been developed.

Thus there exists a need in the art to develop high-throughput screening methods to identify compounds that have the ability to bind duplex and triplex polynucleotides.

### BRIEF SUMMARY OF THE INVENTION

Methods are provided for identifying a polynucleotide complex binding compound that stabilizes or destabilizes a duplex polynucleotide complex comprising the steps of: a) contacting a test compound with (i) a first functionalized nanoparticle having a first oligonucleotide attached thereto and (ii) a second functionalized particle having a second oligonucleotide attached thereto, under conditions that permit hybridization between the first oligonucleotide and the second oligonucleotide to form a duplex polynucleotide complex, and b) identifying the test compound as a duplex polynucleotide complex binding compound that stabilizes or destabilizes the duplex polynucleotide complex when melting temperature of the duplex polynucleotide complex in the presence of the test compound differs from melting temperature of the duplex polynucleotide complex in the absence of the test compound. In another aspect, of the methods, the first oligonucleotide attached to the first nanoparticle and the second oligonucleotide attached to the second nanoparticle are contacted with a free oligonucleotide under conditions that permit formation of a triplex polynucleotide complex, and the test compound is identified as a triplex polynucleotide complex binding compound that stabilizes or destabilizes the triplex polynucleotide complex when melting temperature of the triplex polynucleotide complex in the presence of the test compound differs from melting temperature of the triplex polynucleotide complex in the absence of the test compound.

In one aspect of the methods, an increase in melting temperature of the duplex polynucleotide complex or the triplex polynucleotide complex in the presence of the test compound, compared to melting temperature of the duplex polynucleotide complex or the triplex polynucleotide complex in the absence of the test compound, identifies the test compound as one that stabilizes the duplex polynucleotide complex or the triplex polynucleotide complex.

In another aspect of the methods, a decrease in melting temperature of the duplex polynucleotide complex or the triplex polynucleotide complex in the presence of the test compound, compared to melting temperature of the duplex polynucleotide complex or the triplex polynucleotide complex in the absence of the test compound, identifies the test compound as one that destabilizes the duplex polynucleotide complex or triplex polynucleotide complex.

In various embodiments of the methods, the first oligonucleotide or the second oligonucleotide is DNA, the first oligonucleotide and the second oligonucleotide are DNA, the first oligonucleotide or the second oligonucleotide is a modified polynucleotide, the first oligonucleotide and the second oligonucleotide are modified polynucleotides, the first oligonucleotide, the second oligonucleotide or the free oligonucleotides is DNA, the first oligonucleotide, the second oligonucleotide and the free oligonucleotide are DNA, the first oligonucleotide, the second oligonucleotide or the free oligonucleotide is a modified polynucleotide, the first oligonucleotide, the second oligonucleotide and the free oligonucleotide are modified polynucleotides.

In one aspect of the methods, the first nanoparticle and the second nanoparticle are gold nanoparticles.

In one embodiment, formation of the duplex polynucleotide complex or the triplex polynucleotide complex is detected by a red-to-blue color change associated with aggregation of the first gold nanoparticle with the second gold nanoparticle. And in one aspect, the color change is detected without instrumentation. In another embodiment, formation of the duplex polynucleotide complex or the triplex polynucleotide complex is detected by decreased plasmin resonance associated with aggregation of the first gold nanoparticle with the second gold nanoparticle.

Also provided are methods to determine the relative strength of a test duplex polynucleotide complex binding compound compared to a control duplex polynucleotide complex binding compound, comprising the step of: comparing melting temperature of a duplex polynucleotide complex formed between a first oligonucleotide attached to a first nanoparticle and a second oligonucleotide attached to a second nanoparticle and further including the test compound, to melting temperature of a duplex polynucleotide complex formed between the first oligonucleotide attached to the first nanoparticle and the second oligonucleotide attached to the second nanoparticle and further including the control compound, wherein (i) a higher melting temperature in the presence of the test compound compared to melting temperature in the presence of the control compound indicates stronger binding of the test compound than binding of the control compound, (ii) a lower melting temperature in the presence of the test compound compared to melting temperature in the presence of the control compound indicates weaker binding of the test compound than binding of the control compound, (iii) and melting temperature in the presence of the control compound essentially equal to melting temperature in the presence of the control compound indicates essentially equal binding of the test compound and the control compound. In various aspects, the first oligonucleotide or the second oligonucleotide is DNA, the first oligonucleotide and the second oligonucleotide are DNA, the first oligonucleotide or the second oligonucleotide is a modified polynucleotide, the first oligonucleotide and the second oligonucleotide are modified polynucleotides.

Also provided are methods to determine the relative strength of a test triplex polynucleotide complex binding compound compared to a control binding compound, comprising the step of: comparing melting temperature of a triplex polynucleotide complex formed between a first oligonucleotide attached to a first nanoparticle, a second oligonucleotide attached to a second nanoparticle and a free oligonucleotide and further including a test compound, to melting temperature of a triplex polynucleotide complex formed between the first oligonucleotide, the second oligonucleotide, and the free oligonucleotide and further including the control compound, wherein (i) a higher melting temperature in the presence of the test compound compared to melting temperature in the presence of the control compound indicates stronger binding of the test compound than binding of the control compound, (ii) a lower melting temperature in the presence of the test compound compared to melting temperature in the presence of the control compound indicates weaker binding of the test compound than binding of the control compound, (iii) and melting temperature in the presence of the control compound essentially equal to melting temperature in the presence of the control compound indicates essentially equal binding of the test compound and the control compound.

In various embodiments, the first oligonucleotide, the second oligonucleotide or the free oligonucleotides is DNA, the first oligonucleotide, the second oligonucleotide and the free oligonucleotide are DNA, the first oligonucleotide, the second oligonucleotide or the free oligonucleotide is a modified polynucleotide, the first oligonucleotide, the second oligonucleotide and the free oligonucleotide are modified polynucleotides.

In one aspect, the first nanoparticle and the second nanoparticle are gold nanoparticles.

In one embodiment, formation of the duplex polynucleotide complex or the triplex polynucleotide complex is detected by a red-to-blue color change associated with aggregation of the first gold nanoparticle with the second gold nanoparticle, and in one aspect, the color change is detected without instrumentation.

In another embodiment, formation of the duplex polynucleotide complex or the triplex polynucleotide complex is detected by decreased plasmin resonance associated with aggregation of the first gold nanoparticle with the second gold nanoparticle.

The methods of the invention are also contemplated to be performed in a high throughput format.

In another embodiment, methods are provided for identifying a polynucleotide complex binding compound that stabilizes or destabilizes a duplex polynucleotide complex comprising the steps of: a) contacting a test compound with (i) a first oligonucleotide immobilized on a substrate and (ii) a functionalized particle having a second oligonucleotide attached thereto, under conditions that permit hybridization between the first oligonucleotide and the second oligonucleotide to form a duplex polynucleotide complex, and b) identifying the test compound as a duplex polynucleotide complex binding compound that stabilizes or destabilizes the duplex polynucleotide complex when melting temperature of the duplex polynucleotide complex in the presence of the test compound differs from melting temperature of the duplex polynucleotide complex in the absence of the test compound. In an alternative aspect of this method, the first oligonucleotide immobilized on the substrate and the second oligonucleotide attached to the nanoparticle are contacted with a free oligonucleotide under conditions that permit formation of a triplex polynucleotide complex, and the test compound is identified as a triplex polynucleotide complex binding compound that stabilizes or destabilizes the triplex polynucleotide complex when melting temperature of the triplex polynucleotide complex in the presence of the test compound differs from melting temperature of the triplex polynucleotide complex in the absence of the test compound.

In Methods in this embodiment an increase in melting temperature of the duplex polynucleotide complex or the triplex polynucleotide complex in the presence of the test compound, compared to melting temperature of the duplex polynucleotide complex or the triplex polynucleotide complex in the absence of the test compound, identifies the test compound as one that stabilizes the duplex polynucleotide complex or the triplex polynucleotide complex. Alternatively, a decrease in melting temperature of the duplex polynucleotide complex or the triplex polynucleotide complex in the presence of the test compound, compared to melting temperature of the duplex polynucleotide complex or the triplex polynucleotide complex in the absence of the test compound, identifies the test compound as one that destabilizes the duplex polynucleotide complex or triplex polynucleotide complex.

In various aspects, the first oligonucleotide or the second oligonucleotide is DNA, the first oligonucleotide and the second oligonucleotide are DNA, the first oligonucleotide or the second oligonucleotide is a modified polynucleotide, the first oligonucleotide and the second oligonucleotide are modified polynucleotides, the first oligonucleotide, the second oligonucleotide or the free oligonucleotides is DNA, the first oligonucleotide, the second oligonucleotide and the free oligonucleotide are DNA, the first oligonucleotide, the second oligonucleotide or the free oligonucleotide is a modified polynucleotide, or the first oligonucleotide, the second oligonucleotide and the free oligonucleotide are modified polynucleotides.

In one aspect, the nanoparticle is a gold nanoparticle. In another aspect, the substrate is a chip, and in one embodiment, the first oligonucleotide is arrayed on the chip.

Also provided are methods to determine the relative strength of a test duplex polynucleotide complex binding compound compared to a control duplex polynucleotide complex binding compound, comprising the step of: comparing melting temperature of a duplex polynucleotide complex formed between a first oligonucleotide immobilized on a substrate and a second oligonucleotide attached to a nanoparticle and further including the test compound, to melting temperature of a duplex polynucleotide complex formed between the first oligonucleotide attached to the first nanoparticle and the second oligonucleotide attached to the second nanoparticle and further including the control compound, wherein (i) a higher melting temperature in the presence of the test compound compared to melting temperature in the presence of the control compound indicates stronger binding of the test compound than binding of the control compound, (ii) a lower melting temperature in the presence of the test compound compared to melting temperature in the presence of the control compound indicates weaker binding of the test compound than binding of the control compound, (iii) and melting temperature in the presence of the control compound essentially equal to melting temperature in the presence of the control compound indicates essentially equal binding of the test compound and the control compound.

In various embodiments, the first oligonucleotide or the second oligonucleotide is DNA, the first oligonucleotide and the second oligonucleotide are DNA, the first oligonucleotide or the second oligonucleotide is a modified polynucleotide, or the first oligonucleotide and the second oligonucleotide are modified polynucleotides.

Also provided are methods to determine the relative strength of a test triplex polynucleotide complex binding compound compared to a control binding compound, comprising the step of: comparing melting temperature of a triplex polynucleotide complex formed between a first oligonucleotide immobilized on a substrate, a second oligonucleotide attached to a nanoparticle and a free oligonucleotide and further including a test compound, to melting temperature of a triplex polynucleotide complex formed between the first oligonucleotide, the second oligonucleotide, and the free oligonucleotide and further including the control compound, wherein (i) a higher melting temperature in the presence of the test compound compared to melting temperature in the presence of the control compound indicates stronger binding of the test compound than binding of the control compound, (ii) a lower melting temperature in the presence of the test compound compared to melting temperature in the presence of the control compound indicates weaker binding of the test compound than binding of the control compound, (iii) and melting temperature in the presence of the control compound essentially equal to melting temperature in the presence of the control compound indicates essentially equal binding of the test compound and the control compound.

In various aspects, the first oligonucleotide, the second oligonucleotide or the free oligonucleotides is DNA, the first oligonucleotide, the second oligonucleotide and the free oligonucleotide are DNA, the first oligonucleotide, the second oligonucleotide or the free oligonucleotide is a modified polynucleotide, the first oligonucleotide, the second oligonucleotide and the free oligonucleotide are modified polynucleotides, the first nanoparticle and the second nanoparticle are gold nanoparticles.

In one aspect, the substrate is a chip, and in one embodiments, the first oligonucleotide is arrayed on the chip.

In another aspect, the methods are high through-put assay methods.

### DESCRIPTION OF THE DRAWINGS

Scheme 1—Representation of structure and color change of nanoassembly in the presence of triplex binder at room temperature

FIG. 1(A) The hybridization kinetics monitored at 520 nm without stirring of NP-1 and NP-2 (1-5 nM each) in the presence DNA-3 (150 nM) (red) and DNA-3+BePI (5 μM) (black). B) The UV spectrum of NP-1 and NP-2 after six hours incubation (1.5 nM each).

FIG. 1(B)—The UV spectrum of NP-1 and NP-2 after 6 hr incubation

FIG. 2(A)—Melting curves of NP-1, NP-2 and DNA-3 assemblies in the presence of DNA binders.

FIG. 2(B)—Melting curves of DNA-1, DNA-2 and DNA-3 (no nanoparticles) in the presence of DNA binders.

FIG. 3—The color change of nanoassembly (NP-1 and NP-2, and DNA-3) in the absence and presence of DNA binders at room temperature.

Scheme 2—Schematic representation of structure and color change of nanoparticle/intercalator assemblies at a specific temperature.

FIG. 4—Melting curves of A) DNA-1 and DNA-2 (no nanoparticle) and B) NP-1 and NP-2 assemblies in the absence of intercalator (black) and the presence of DAPI (red).

FIG. 5—The color chance of nanoassembly (NP-1 and NP-2, each 1.5 nM) in the absence and presence of intercalator (5 μM) at specific temperature.

Scheme 3—Scanometric detection of duplex DNA binders on a chip surface.

FIG. 6—Light scattering from silver enhancement visualized by scanner.

Scheme 4—Scanometric detection of triplex DNA binders on a chip.

FIG. 7—Relative strength of triplex DNA binders at varying salt concentrations.

### DETAILED DESCRIPTION OF THE INVENTION

Methods are provided utilizing polynucleotide functionalized nanoparticles as an integral component for colorimetric-based detection of duplex and triplex polynucleotide binding compounds (alternatively and interchangeably referred to herein as “binders”). Assays contemplated are based on a perturbation of the duplex and triplex polynucleotide melting temperature (Tₘ) induced by the presence of duplex and triplex polynucleotide binders. Strong binding compounds result in a greater increase in Tₘ whereas weaker binders have very little effect on the Tₘ. Alternatively, interaction by other binding compounds result in a decrease in Tₘ. The polynucleotide functionalized nanoparticles further increase the perturbation induced by the binders, thereby expanding the window through which to screen for duplex and triplex polynucleotide binders. These assays demonstrate excellent discrimination between strong, intermediate and weak duplex and triplex polynucleotide binders. This result is due to both the increased perturbation of the Tₘ and to the sharp melting transitions associated with polynucleotide functionalized nanoparticles.

Thus, methods are provided wherein polynucleotide functionalized nanoparticles are used to screen for candidate compounds that bind a polynucleotide complex, methods which are readily adaptable for use in a high-throughput applicable fashion. As used herein, a “polynucleotide complex” is either a double-strand (or duplex) complex or a triple-strand (or triplex) complex. In general, assays used to screen for compounds that interact with a double-strand polynucleotide complex, a first functionalized nanoparticle having a first single-strand polynucleotide attached thereto is contacted with a second functionalized nanoparticle having a second single-strand polynucleotide attached thereto under conditions that allow the first single-strand polynucleotide and the second single-strand polynucleotide to form a double-strand polynucleotide complex. In general, assays used to screen for compounds that interact with a triplex polynucleotide complex, a first functionalized nanoparticles having a first single-strand polynucleotide attached thereto, a second functionalized nanoparticle having a second single-strand polynucleotide attached thereto, and a free single-strand polynucleotide are contacted under conditions that allow the first single-strand polynucleotide, the second single-strand polynucleotide and the free single-stranded polynucleotide to form a triplex polynucleotide complex. Candidate compounds are identified in the methods that promote complex formation or disrupt complex formation. Compounds that promote complex formation either increase the kinetics of polynucleotide hybridization, i.e., complex formation, or increase stability of a formed complex. Compounds that disrupt complex formation decrease the kinetics of complex formation, or preclude significant complex formation or preclude complex formation in its entirety. Methods provided also allow for determining relative binding affinity of candidate compounds for complex formation, relative ability to stabilize complex formation, or relative ability to destabilize complex formation.

In another aspect, methods are provided for determining the binding strength of a candidate compound to a polynucleotide complex wherein a change in the level of complex formed is monitored over changes in conditions under which the polynucleotide complex is formed. In this aspect, the methods allow discrimination between weak, intermediate, and strong polynucleotide complex-binding compounds. Conditions that can be modified include, for example, temperature, pH, salt concentration and concentration of the candidate compound.

In one embodiment, a calorimetric assay is provided based on a detectable color change that occurs when functionalized nanoparticles are brought into proximity with each other as a result of polynucleotide complex formation, wherein a first single-stranded polynucleotide functionalized on a first nanoparticle hybridizes to a second single-stranded polynucleotide functionalized on a second nanoparticle, with (i.e., triplex polynucleotide complex formation) or without (i.e., duplex polynucleotide complex formation) further association of a free single-strand polynucleotide to the complex. In another embodiment, a calorimetric assay is provided that is based on a detectable color change that occurs when functionalized nanoparticles in a polynucleotide complex are removed from proximity to each other, i.e., the polynucleotide complex is disrupted or destabilized and some or all of the nanoparticles in proximity as a result of polynucleotide hybridization are removed from proximity to each other to a degree sufficient to cause a color change. In one aspect, the calorimetric readout can be visualized with the naked eye without resorting to additional instrumentation, and in this aspect, the simplicity of the assay makes it more convenient than other methods such as mass spectroscopy, nuclear magnetic resonance, light scattering, electrochemistry, and fluorometry, competitive dialysis, electrophoresis and ultraviolet/visible (UV/Vis) melting experiments known in the art. All methods provided are easily adapted for high-throughput screening which can be used, for example, to identify potentially useful compounds from large combinatorial libraries based on their interaction with duplex or triplex polynucleotide complexes.

### A. Intercalator Compounds

In one aspect, the candidate compound is an intercalator. “Intercalation” refers to the reversible or irreversible inclusion of, for example, a molecule between two other molecules. Of significance to the present methods, a large class of molecules, mostly polycyclic, aromatic, and planar, are known that intercalate into a double-strand polynucleotide between two adjacent base pairs. Examples of intercalators known in the art include ethidium, proflavin, daunomycin, doxorubicin, and thalidomide. Intercalators in general induce local structural changes into the double-stranded polynucleotide which lead to functional changes, often the inhibition of transcription and replication processes which makes intercalators mutagenic. Intercalators have the property of (i) interacting with a double-strand polynucleotide complex without affecting stability of the hybridized complex, (ii) interacting with a double-strand polynucleotide complex and increasing stability of the hybridized complex, or (iii) interacting with the double-strand polynucleotide complex and decreasing stability of the hybridized complex. Still other intercalators have the ability to interact with bases in a single-strand polynucleotide and prevent polynucleotide complex formation. While not technically “intercalators” in the sense that it interacts with hybridized polynucleotides, compounds of this type are readily identified in methods provided.

In one embodiment, the intercalator recognizes and binds to a polynucleotide complex formed between a first oligonucleotide and a second oligonucleotide. In another embodiment, the interacting compound promotes complex formation between a first oligonucleotide and a second oligonucleotide or stabilizes a complex formed between the first and second oligonucleotides. Alternatively, the intercalator destabilizes a complex formed between the first oligonucleotide and the second oligonucleotide.

Regardless of the action of the interacting compound, the first oligonucleotide and the second oligonucleotide are, in general, sufficiently complementary to hybridize to each other, or can be forced to hybridize through environmental modification, and complex formation results from hybridization between said first oligonucleotide and said second oligonucleotide.

In one aspect, compounds recognize and bind to a first and second oligonucleotide complex and do not have any effect on promoting complex formation or stabilizing or destabilizing an existing complex. Compounds of this type are no effect on the kinetics of oligonucleotide association and/or dissociation, and while compounds of this type are not generally detectable in an assay carried out in their presence and absence, they are detectable when used in assays that determine relative binding of this type of compound compared to other compounds that do have an effect on polynucleotide complex formation, stabilization, disruption and/or destabilization. For compounds that promote complex formation between a first oligonucleotide and a second oligonucleotide, intercalation increases the kinetics of oligonucleotide association, with or without affecting the opposing rate of oligonucleotide dissociation, thereby promoting hybridization. In another aspect, a compound that stabilizes a hybridization complex, i.e., allows formation of a more stable polynucleotide complex, is one that slows the kinetics of oligonucleotide dissociation, regardless of the effects of the compound on the kinetics of association. In other words, stabilization inhibits dissociation. In still other aspects, compounds interact with a polynucleotide complex and destabilize the complex, and in still other aspects, compounds reduce or prevent complex formation.

A “more stable complex” is defined as one that melts at a higher temperature in the presence of the intercalator than in the absence of the intercalator. Conversely, a “less stable complex” is defined as one that melts at a lower temperature in the presence of the intercalator than in the absence of the intercalator. The term “melts” is understood in the art to mean dissociation of hybridized polynucleotides, generally brought about by an increase in temperature to greater than a “melting temperature, Tₘ.” Changes in environmental conditions can alter the Tₘ for any given hybridization complex, such conditions including for example, pH, salt concentration, and the concentration of other hybridization mixture additives known in the art.

Thus, methods provided are carried out in various ways depending on the properties of the compounds being screened. In one aspect, the first polynucleotide attached to the first nanoparticle and the second polynucleotide attached to the second nanoparticle are contacted under conditions that allow the first polynucleotide and the second polynucleotide to hybridize. After hybridization has occurred, one or more test compounds are added to mixture. Alternatively, the first polynucleotide, the second polynucleotide and one or more test compounds are combined at the same time. In another aspect, the first polynucleotide attached to the first nanoparticle and one or more test compounds are contacted prior to addition of the second polynucleotide attached to the second nanoparticle, and in still another aspect, one or more test compounds in the reaction mixture are contacted with the first polynucleotide on the first nanoparticle and the second polynucleotide attached to the second nanoparticle which are added to the reaction mixture at the same time. Regardless of the order in which the polynucleotides attached to the nanoparticle and the one or more test compounds are contacted, the assay optionally continues with alteration of one or more environmental conditions which, in the absence of a test compound, would normally result in an increased or decreased level, or degree, of polynucleotide complex in the mixture. Environmental changes include without limitation change in temperature, change in salt concentration, change in pH, change in concentration of a compound, such as without limitation, formamide, that lowers Tₘ of a hybridization complex compared to Tₘ in its absence. Whether the test compound has an effect on the level or degree of polynucleotide complex in the mixture is determined by comparison to a control assay carried out under identical conditions but without the test compound.

### B. Triplex Binders

In another embodiment, the polynucleotide complex-binding compound is a triplex-binding compound, or triplex binder. “Triplex binders” as used herein are compounds that recognize and bind to triple helix polynucleotide complexes consisting of three polynucleotide strands. In general, a triple helix forms when a third polynucleotide binds in the major groove of a double-strand polynucleotide complex. Triplex binders include compounds that intercalate into triple helix, interacting with one, two or three polynucleotides in the helix, as discussed herein for intercalators into a double-strand polynucleotide complex, as well as compounds that recognize and bind to more exterior regions of the helix, i.e., that do not intercalate into the helix.

For identification of compounds of this type, the contacting step in the method is carried out in the presence of a first functional nanoparticle having a first oligonucleotide attached thereto, a second functionalized nanoparticle having a second oligonucleotide attached thereto, and a free, single-strand polynucleotide, i.e., “free” in that the single-strand polynucleotide is not attached to a nanoparticle, and the complex formed between the first oligonucleotide and the second oligonucleotide further comprises the “free” single-strand polynucleotide

In this aspect of the methods provided, the first oligonucleotide, the second oligonucleotide and the free oligonucleotide are sufficiently complementary to hybridize to provide a triple-strand polynucleotide complex. The order in which the oligonucleotide components of the complex associate to provide the triplex complex is irrelevant.

As with intercalator compounds, in one aspect the triplex binding compound promotes triplex complex formation. In this embodiment, the presence of the triplex binder increases the kinetics of triplex complex formation, compared to triplex complex formation in the absence of the triplex binder, with or without an effect on the rate of triplex complex dissociation. A triplex binding compound of this type is in general part of the triplex complex, however, there are aspects of the methods wherein the compound that promotes complex formation does so by way of inhibiting activity of yet another compound that inhibits complex formation. A compound that acts in this way is not a triplex binding compound per se, but is a promoter of triplex complex formation and can be identified by the methods provided in the presence of the putative inhibitor of complex formation.

In another embodiment, the triplex binding compound stabilizes an existing triplex complex and in another aspect, the triplex binding compound destabilizes an existing complex. Like intercalator compounds described herein, triplex binding compounds of this type alter the Tₘ of the triplex complex. While it is generally understood in the art that Tₘ is used for describing the stability of hybridized, double-strand polynucleotide complexes, the term also applies to triple-strand, or triplex polynucleotides which can, for example, be subjected to a change in an environmental condition that causes the complex polynucleotides to dissociate, whether it is a single polynucleotide strand that dissociates from two remaining associated polynucleotides or whether all three polynucleotides dissociate.

Thus, as discussed herein, the order in which the first polynucleotide attached to the first nanoparticle, the second polynucleotide attached to the second nanoparticle, the free polynucleotide and one or more test compounds are added to the reaction mixture is determined based on the properties of the test compound being examined. In one aspect, all three polynucleotides are first contacted, followed by addition of a test compound to the mixture. In another aspect, two polynucleotides are contacted, a test compound is added to the mixture, and the third polynucleotide is added. In another aspect, a single polynucleotide is contacted with a test compound, after which one or both of the other two polynucleotides are added to the mixture. In still another aspect, all three polynucleotides are introduced into a reaction mix containing a test compound. In yet another aspect, the three polynucleotides are introduced individually in any order into a reaction mixture containing a test compound. In still another aspect, a single polynucleotide is introduced into a reaction mixture containing a test compound, followed by introduction of the remaining two polynucleotides.

Also as discussed herein, regardless of the order in which the polynucleotides and the one or more test compounds are contacted, the assay optionally continues with alteration of one or more environmental conditions which, in the absence of a test compound, would normally result in an increased or decreased level, or degree, of polynucleotide complex in the mixture. Environmental changes include without limitation change in temperature, change in salt concentration, change in pH, change in concentration of a compound, such as without limitation, formamide, that lowers Tₘ of a hybridization complex compared to Tₘ in its absence. Whether the test compound has an effect on the level or degree of polynucleotide complex in the mixture is determined by comparison to a control assay carried out under identical conditions but without the test compound.

### C. Chip-Based Assay

In another aspect, methods are provided wherein a polynucleotide attached to a substrate and a polynucleotide functionalized on a nanoparticle are contacted with a candidate compound under conditions that allow the polynucleotide on the substrate and the polynucleotide on the nanoparticle to hybridize to form a polynucleotide complex, and the candidate compound is identified as a polynucleotide complex binding compound wherein melting temperature of the polynucleotide complex in the presence of the compound differs from melting temperature of the polynucleotide complex in the absence of the compound. Methods in this aspect are performed with the polynucleotide on the substrate and the polynucleotide on the nanoparticle alone which will form a double strand polynucleotide complex, or also in the presence of a free polynucleotide, i.e., a polynucleotide not attached to either a substrate or a nanoparticle, under conditions that allow the polynucleotide on the substrate, the polynucleotide on the nanoparticle, and the free polynucleotide to form a triple strand, or triplex, polynucleotide complex.

In one aspect of this embodiment, the substrate is a chip and the polynucleotide on the chip substrate is arrayed. Polynucleotide chips are well known in the art. Use of a chip array allows for multiple polynucleotides to be used in a screening process at one time, wherein the polynucleotides on the array have the same sequence, similar sequences, or different sequences. Methods for attaching a polynucleotide to a substrate, and in particular a chip substrate, are well known and routinely practiced in the art.

A chip based assay which relies on polynucleotide functionalized nanoparticles incorporates high discrimination capabilities introduced by these materials while simultaneously increasing the high-throughput capabilities and reducing sample consumption. The scanometric detection process is a modification of a previously reported method well known in the art. Polynucleotides are first arrayed on a glass chip. Nanoparticles which are functionalized with a polynucleotide sufficiently complementary to the arrayed polynucleotide are hybridized to the chip in the presence of different polynucleotide complex binding test compounds, or in the alternative, a test compound is added after a time that hybridization is at equilibrium. Hybridization is followed by a silver enhancement process which increases the light scattering of the nanoparticles. The light scattering signal from the silver enhanced nanoparticles is visualized using, for example, a Verigene ID scanner. To elucidate strong, intermediate and weak complex binders, the hybridization process is performed at more stringent conditions by increasing the temperature, decreasing the salt concentration, changing pH or any other change in environmental condition as discussed herein. As conditions become more stringent, polynucleotide interaction requires complex binding compounds (binders) to increase the stability of the interacting polynucleotides in order for the nanoparticles to remain intact on the chip surface. A reduction of nanoparticles on the chip surface directly correlates with a reduction of signal post silver enhancement, thereby differentiating polynucleotide binding compounds of different strengths.

Thus, methods are provided wherein one polynucleotide which, in the methods described herein is attached to a nanoparticle, is in this aspect attached to a substrate and hybridization of a polynucleotide on a substrate and a polynucleotide attached to a nanoparticle provides the duplex polynucleotide complex. Methods in this aspect optionally include a “free” polynucleotide, as described herein, when a test compound is being assayed for its ability to interact with a triplex polynucleotide complex.

In one aspect, the polynucleotide on the substrate is initially in the reaction mixture alone, after which (i) a test compound is added, followed by addition of one or two polynucleotides together which form the polynucleotide complex, (ii) a second polynucleotide is added, followed by a test compound, and optionally followed by a third polynucleotide, or (iii) a second polynucleotide and a third polynucleotide are added, followed by addition of a test compound. In methods including a second and a third polynucleotide, it will be understood that one polynucleotide is attached to a nanoparticle and the other is a free polynucleotide. Depending on the assay, environmental conditions are modified as described herein at any time during the assay

### D. Candidate Compounds

Methods provided allow for identification of previously unknown polynucleotide complex binding compounds, as well as characterization of known compounds with respect to their binding properties as discussed herein. In various embodiments, the interacting compound is for example, and without limitation, a polynucleotide or modification thereof as described herein, a small molecule, a peptide, or a protein. Polynucleotide candidate compounds include any or all types of polynucleotides described as capable of being functionalized onto a nanoparticle. Various other polynucleotide complex binding compounds useful in practice of the methods are discussed below without limitation, and the worker of ordinary skill will readily appreciate that the methods provided are amendable for testing any compound.

1. Small Molecules

In one aspect, methods are provided to identify a polynucleotide complex-interacting molecule which is a small molecule. As is understood in the art, the term “small molecule” includes organic and inorganic compounds which are either naturally-occurring compounds, modifications of naturally-occurring compounds, or synthetic compounds. In one aspect, individual small molecules are employed in the methods, i.e., individual candidate compounds are screened one at a time for their ability to interact with a polynucleotide complex. In another aspect, libraries of small molecules (or subsets of a library), or any group of compounds, are screened at once. When a library (or subset thereof) is screened, polynucleotide complex interaction is assessed for a pool of small molecules which includes at least two different candidate small molecule compounds. If polynucleotide interaction is detected for a pool, wherein at least one member of the pools gives rise to the interaction detected, the pool is deconvoluted, i.e., the members of the pool are separated either individually or into smaller subpools, and the screening process is continued until the member or members of the original pool that interact with the polynucleotide complex is/are identified.

In one aspect, small molecule libraries are synthesized according to methods well known and routinely practiced in the art. See, for example, Thompson and Ellman, Chem. Rev. 1996, 96, 555-600, Shipps, et al., Proc. Natl. Acad. Sci. USA, Vol. 94, pp. 11833-11838, October 1997, and Combinatorial Library Design and Evaluation—Principles, Software Tools and Applications in Drug Discovery, Ghose and Viswanadhan (eds), Marcel Dekker 2001. Alternatively, small libraries are obtained from any of a number of sources including, for example, the NIH Molecular Libraries Small Molecule Repository. Alternative sources include AnalytiCon Discovery GmbH (Potsdam, Germany) which makes available MEGAbolite®, pure natural product small molecule libraries and NatDiverse™, semi-synthetic natural product analogue small molecule libraries; Quantum Pharmaceuticals Ltd. (Moscow, Russian Federation); and Praecis Pharmaceuticals Incorporated (Waltham, Mass.).

Still other binding compounds include intercalators produced by Imenik Instituta Ru d̅er Bo\check{s}ković, such as cyclic and acyclic bisphenanthridinium derivatives, 4,9-Diazapyrenium systems, and phenanthridinium-nucleobase conjugates, as well as triplex binders as described in Diego, et al., Nucleic Acids Res. 2000 May 15; 28(10): 2128-2134.

2. Peptides

Peptides contemplated for use in the methods provided include those derived from commercially available sources such as Dyax Corp. (Cambridge, Mass.). Dyax libraries include structured peptide libraries comprising small, disulfide-constrained cyclic peptide compounds that range in size from six to twelve amino acids, wherein the number of distinct peptide structures in each library typically exceeds 1 billion; (ii) linear peptide libraries wherein 19 amino acids (no cysteine) at each position in a 20-mer peptide are allowed to create a library of 10 billion peptides; (iii) substrate phage peptide libraries wherein all 19 amino acids (no cysteine) at each position in a 13-mer peptide are allowed to create a library of approximately 100 million peptides.

Still other commercially available peptide libraries include those from Peptide libraries Eurogentec s.a. (Belgium) and Cambridge Peptide (Cambridge, UK).

Preparation of Peptide Libraries Useful in Practice of the Method is Well Known in the art, as described by Jung (ed) Combinatorial Peptide and Nonpeptide Libraries: A Handbook and in Devlin, et al., Science, Vol 249, Issue 4967, 404-406, as well from use of commercially available synthesis kits from, for example, Sigma-Genosys.

3. Proteins

Proteins contemplated for use in the methods provided include those derived from synthesized proteins libraries as described in Matsuura, et al., Protein Science (2002), 11:2631-2643, Ohuchi, et al., Nucleic Acids Res. 1998 October 1; 26(19): 4339-4346, WO/1999/011655, WO/1998/047343, U.S. Pat. No. 6,844,161 and U.S. Pat. No. 6,403,312. Commercially available kits for production of protein libraries are also know in the art and available from, for example, BioCat GmbH (Heidelberg).

Protein libraries useful in practice of the methods are also commercially available from, for example, Dyax Corp. (Cambridge, Mass.).

### E. Complex Detection

Regardless of the type of interacting compound being identified, methods are provided wherein polynucleotide complex formation is detected by an observable change. In one aspect, complex formation gives rise to a color change which is observed with the naked eye or spectroscopically. When using gold nanoparticles, a red-to-blue color change occurs with nanoparticle aggregation which often is detected with the naked eye. In another aspect, polynucleotide complex formation gives rise to aggregate formation which is observed by electron microscopy or by nephelometry. Aggregation of nanoparticles in general gives rise to decreased plasmon resonance. In still another aspect, complex formation gives rise to precipitation of aggregated nanoparticles which is observed with the naked eye or microscopically.

The observation of a color change with the naked eye is, in one aspect, made against a background of a contrasting color. For instance, when gold nanoparticles are used, the observation of a color change is facilitated by spotting a sample of the hybridization solution on a solid white surface (such as, without limitation, silica or alumina TLC plates, filter paper, cellulose nitrate membranes, nylon membranes, or a C-18 silica TLC plate) and allowing the spot to dry. Initially, the spot retains the color of the hybridization solution, which ranges from pink/red, in the absence of hybridization, to purplish-red/purple, if there has been hybridization. On drying at room temperature or 80° C. (temperature is not critical), a blue spot develops if the nanoparticle-oligonucleotide conjugates had been linked by hybridization prior to spotting. In the absence of hybridization, the spot is pink. The blue and the pink spots are stable and do not change on subsequent cooling or heating or over time providing a convenient permanent record of the test. No other steps (such as a separation of hybridized and unhybridized nanoparticle-oligonucleotide conjugates) are necessary to observe the color change.

An alternate method for visualizing the results from practice of the methods is to spot a sample of nanoparticle probes on a glass fiber filter (e.g., Borosilicate Microfiber Filter, 0.7 micron pore size, grade FG75, for use with gold nanoparticles 13 nm in size), while drawing the liquid through the filter. Subsequent rinsing washes the excess, non-hybridized probes through the filter, leaving behind an observable spot comprising the aggregates generated by hybridization of the nanoparticle probes (retained because these aggregates are larger than the pores of the filter). This technique allows for greater sensitivity, since an excess of nanoparticle probes can be used.

Depending on experimental design, obtaining a detectable change depends on hybridization of different oligonucleotides, i.e., double-strand or triple-strand polynucleotide complex formation, or disassociation of hybridized oligonucleotides, i.e., complex disassociation. Mismatches in oligonucleotide complementarity decrease the stability of the complex. It is well known in the art that a mismatch in base pairing has a much greater destabilizing effect on the binding of a short oligonucleotide probe than on the binding of a long oligonucleotide probe.

In other embodiments, the detectable change is created by labeling the oligonucleotides, the nanoparticles, or both with molecules (e.g., and without limitation, fluorescent molecules and dyes) that produce detectable changes upon hybridization of the oligonucleotides on the nanoparticles. In one aspect, oligonucleotides functionalized on nanoparticles have a fluorescent molecule attached to the terminus distal to the nanoparticle attachment terminus. Metal and semiconductor nanoparticles are known fluorescence quenchers, with the magnitude of the quenching effect depending on the distance between the nanoparticles and the fluorescent molecule. In the single-strand state, the oligonucleotides attached to the nanoparticles interact with the nanoparticles, so that significant quenching is observed. Upon polynucleotide complex formation, the fluorescent molecule will become spaced away from the nanoparticles, diminishing quenching of the fluorescence. Longer oligonucleotides give rise to larger changes in fluorescence, at least until the fluorescent groups are moved far enough away from the nanoparticle surface so that an increase in the change is no longer observed. Useful lengths of the oligonucleotides can be determined empirically. Thus, in various aspects, metallic and semiconductor nanoparticles having fluorescent-labeled oligonucleotides attached thereto are used in any of the assay formats described herein.

Methods of labeling oligonucleotides with fluorescent molecules and measuring fluorescence are well known in the art. Suitable fluorescent molecules are also well known in the art and include without limitation fluoresceins, rhodamines and Texas Red.

In yet another embodiment, two types of fluorescent-labeled oligonucleotides attached to two different particles can be used. Suitable particles include polymeric particles (such as, without limitation, polystyrene particles, polyvinyl particles, acrylate and methacrylate particles), glass particles, latex particles, Sepharose beads and others like particles well known in the art. Methods of attaching oligonucleotides to such particles are well known and routinely practiced in the art. See Chrisey et al., Nucleic Acids Research, 24, 3031-3039 (1996) (glass) and Charreyre et al., Langmuir, 13, 3103-3110 (1997), Fahy et al., Nucleic Acids Research, 21, 1819-1826 (1993), Elaissari et al., J. Colloid Interface Sci., 202, 251-260 (1998), Kolarova et al., Biotechniques, 20, 196-198 (1996) and Wolf et al., Nucleic Acids Research, 15, 2911-2926 (1987) (polymer/latex). In particular, a wide variety of functional groups are available on the particles or can be incorporated into such particles. Functional groups include carboxylic acids, aldehydes, amino groups, cyano groups, ethylene groups, hydroxyl groups, mercapto groups, and the like. Nanoparticles, including metallic and semiconductor nanoparticles, can also be used.

In aspects wherein two fluorophores are employed, the two fluorophores are designated “d” and “a” for donor and acceptor. A variety of fluorescent molecules useful in such combinations are well known in the art and are available from, e.g., Molecular Probes. An attractive combination is fluorescein as the donor and Texas Red as acceptor. The two types of nanoparticle-oligonucleotide conjugates with “d” and “a” attached are mixed, and fluorescence measured in a fluorimeter. The mixture is excited with light of the wavelength that excites d, and the mixture is monitored for fluorescence from a. Upon hybridization, “d” and “a” will be brought in proximity. In the case of non-metallic, non-semiconductor particles, hybridization is shown by a shift in fluorescence from that for “d” to that for “a” or by the appearance of fluorescence for “a” in addition to that for “d.” In the absence of hybridization, the fluorophores will be too far apart for energy transfer to be significant, and only the fluorescence of “d” will be observed. In the case of metallic and semiconductor nanoparticles, lack of hybridization will be shown by a lack of fluorescence due to “d” or “a” because of quenching as discussed herein. Hybridization is shown by an increase in fluorescence due to “a.” The person of ordinary skill in the art will readily appreciate that the discussion herein as it relates to formation of a double-strand complex, but that the use of two or three fluorophores can be utilized when a triplex polynucleotide complex is used in the method.

Other labels besides fluorescent molecules can be used, such as chemiluminescent molecules, which will give a detectable signal or a change in detectable signal upon hybridization.

### F. Nanoparticles

In general, nanoparticles contemplated include any compound or substance with a high loading capacity for an oligonucleotide as described herein, including for example and without limitation, a metal, a semiconductor, and an insulator particle compositions, and a dendrimer (organic or inorganic).

Thus, nanoparticles are contemplated for use in the methods which comprise a variety of inorganic materials including, but not limited to, metals, semi-conductor materials or ceramics as described in US patent application No 20030147966. For example, metal-based nanoparticles include those described herein. Ceramic nanoparticle materials include, but are not limited to, brushite, tricalcium phosphate, alumina, silica, and zirconia. Organic materials from which nanoparticles are produced include carbon. Nanoparticle polymers include polystyrene, silicone rubber, polycarbonate, polyurethanes, polypropylenes, polymethylmethacrylate, polyvinyl chloride, polyesters, polyethers, and polyethylene. Biodegradable, biopolymer (e.g. polypeptides such as BSA, polysaccharides, etc.), other biological materials (e.g. carbohydrates), and/or polymeric compounds are also contemplated for use in producing nanoparticles.

In one embodiment, the nanoparticle is metallic, and in various aspects, the nanoparticle is a colloidal metal. Thus, in various embodiments, nanoparticles useful in the practice of the methods include metal (including for example and without limitation, gold, silver, platinum, aluminum, palladium, copper, cobalt, indium, nickel, or any other metal amenable to nanoparticle formation), semiconductor (including for example and without limitation, CdSe, CdS, and CdS or CdSe coated with ZnS) and magnetic (for example, ferromagnetite) colloidal materials. Other nanoparticles useful in the practice of the invention include, also without limitation, ZnS, ZnO, Ti, TiO₂, Sn, SnO₂, Si, SiO₂, Fe, Fe⁺⁴, Ag, Cu, Ni, Al, steel, cobalt-chrome alloys, Cd, titanium alloys, AgI, AgBr, HgI₂, PbS, PbSe, ZnTe, CdTe, In₂S₃, In₂Se₃, Cd₃P₂, Cd₃As₂, InAs, and GaAs. Methods of making ZnS, ZnO, TiO₂, AgI, AgBr, HgI₂, PbS, PbSe, ZnTe, CdTe, In₂S₃, In₂Se₃, Cd₃P₂, Cd₃As₂, InAs, and GaAs nanoparticles are also known in the art. See, e.g., Weller, Angew. Chem. Int. Ed. Engl., 32, 41 (1993); Henglein, Top. Curr. Chem., 143, 113 (1988); Henglein, Chem. Rev., 89, 1861 (1989); Brus, Appl. Phys. A., 53, 465 (1991); Bahncmann, in Photochemical Conversion and Storage of Solar Energy (eds. Pelizetti and Schiavello 1991), page 251; Wang and Herron, J. Phys. Chem., 95, 525 (1991); Olshavsky, et al., J. Am. Chem. Soc., 112, 9438 (1990); Ushida et al., J. Phys. Chem., 95, 5382 (1992).

In practice, methods are provided using any suitable nanoparticle having oligonucleotides attached thereto that are in general suitable for use in detection assays known in the art to the extent and do not interfere with polynucleotide complex formation, i.e., hybridization to form a double-strand or triple-strand complex. The size, shape and chemical composition of the particles contribute to the properties of the resulting oligonucleotide-functionalized nanoparticle. These properties include for example, optical properties, optoelectronic properties, electrochemical properties, electronic properties, stability in various solutions, magnetic properties, and pore and channel size variation. The use of mixtures of particles having different sizes, shapes and/or chemical compositions, as well as the use of nanoparticles having uniform sizes, shapes and chemical composition, is contemplated. Examples of suitable particles include, without limitation, nanoparticles, aggregate particles, isotropic (such as spherical particles) and anisotropic particles (such as non-spherical rods, tetrahedral, prisms) and core-shell particles such as the ones described in U.S. patent application Ser. No. 10/034,451, filed Dec. 28, 2002 and International application no. PCT/US01/50825, filed Dec. 28, 2002, the disclosures of which are incorporated by reference in their entirety.

Methods of making metal, semiconductor and magnetic nanoparticles are well-known in the art. See, for example, Schmid, G. (ed.) Clusters and Colloids (VCH, Weinheim, 1994); Hayat, M. A. (ed.) Colloidal Gold: Principles, Methods, and Applications (Academic Press, San Diego, 1991); Massart, R., IEEE Transactions On Magnetics, 17, 1247 (1981); Ahmadi, T. S. et al., Science, 272, 1924 (1996); Henglein, A. et al., J. Phys. Chem., 99, 14129 (1995); Curtis, A. C., et al., Angew. Chem. Int. Ed. Engl., 27, 1530 (1988). Preparation of polyalkylcyanoacrylate nanoparticles prepared is described in Fattal, et al., J. Controlled Release (1998) 53: 137-143 and U.S. Pat. No. 4,489,055. Methods for making nanoparticles comprising poly(D-glucaramidoamine)s are described in Liu, et al., J. Am. Chem. Soc. (2004) 126:7422-7423. Preparation of nanoparticles comprising polymerized methylmethacrylate (MMA) is described in Tondelli, et al., Nucl. Acids Res. (1998) 26:5425-5431, and preparation of dendrimer nanoparticles is described in, for example Kukowska-Latallo, et al., Proc. Natl. Acad. Sci. USA (1996) 93:4897-4902 (Starburst polyamidoamine dendrimers).

Suitable nanoparticles are also commercially available from, for example, Ted Pella, Inc. (gold), Amersham Corporation (gold) and Nanoprobes, Inc. (gold).

Also as described in US patent application No 20030147966, nanoparticles comprising materials described herein are available commercially or they can be produced from progressive nucleation in solution (e.g., by colloid reaction), or by various physical and chemical vapor deposition processes, such as sputter deposition. See, e.g., HaVashi, (1987) Vac. Sci. Technol. July/August 1987, A5(4):1375-84; Hayashi, (1987) Physics Today, December 1987, pp. 44-60; MRS Bulletin, January 1990, pgs. 16-47.

As further described in US patent application No 20030147966, nanoparticles contemplated are produced using HAuCl₄ and a citrate-reducing agent, using methods known in the art. See, e.g., Marinakos et al., (1999) Adv. Mater. 11: 34-37; Marinakos et al., (1998) Chem. Mater. 10: 1214-19; Enustun & Turkevich, (1963) J. Am. Chem. Soc. 85: 3317. Tin oxide nanoparticles having a dispersed aggregate particle size of about 140 nm are available commercially from Vacuum Metallurgical Co., Ltd. of Chiba, Japan. Other commercially available nanoparticles of various compositions and size ranges are available, for example, from Vector Laboratories, Inc. of Burlingame, Calif.

### G. Nanoparticle Size

In various aspects, methods provided include those utilizing nanoparticles which range in size from about 1 nm to about 250 nm in mean diameter, about 1 nm to about 240 nm in mean diameter, about 1 nm to about 230 nm in mean diameter, about 1 nm to about 220 nm in mean diameter, about 1 nm to about 210 nm in mean diameter, about 1 nm to about 200 nm in mean diameter, about 1 nm to about 190 nm in mean diameter, about 1 nM to about 180 nM in mean diameter, about 1 nm to about 170 nm in mean diameter, about 1 nm to about 160 nm in mean diameter, about 1 nm to about 150 nm in mean diameter, about 1 nm to about 140 nm in mean diameter, about 1 nm to about 130 nm in mean diameter, about 1 nm to about 120 nm in mean diameter, about 1 nm to about 110 nm in mean diameter, about 1 nm to about 100 nm in mean diameter, about 1 nm to about 90 nm in mean diameter, about 1 nm to about 80 nm in mean diameter, about 1 nm to about 70 nm in mean diameter, about 1 nm to about 60 nm in mean diameter, about 1 nm to about 50 nm in mean diameter, about 1 nm to about 40 nm in mean diameter, about 1 nm to about 30 nm in mean diameter, or about 1 nm to about 20 nm in mean diameter, about 1 nm to about 10 nm in mean diameter. In other aspects, the size of the nanoparticles is from about 5 nm to about 150 nm (mean diameter), from about 5 to about 50 nm, from about 10 to about 30 nm. The size of the nanoparticles is from about 5 nm to about 150 nm (mean diameter), from about 30 to about 100 nm, from about 40 to about 80 nm. The size of the nanoparticles used in a method varies as required by their particular use or application. The variation of size is advantageously used to optimize certain physical characteristics of the nanoparticles, for example, optical properties or amount surface area that can be derivatized as described herein.

### H. Polynucleotide Features

As used herein, the term “polynucleotide,” either functionalized on a nanoparticle or as a candidate compound, is used interchangeably with the term oligonucleotide.

Each nanoparticle utilized in the methods provided has a plurality of oligonucleotides attached to it. As a result, each nanoparticle-oligonucleotide conjugate has the ability to hybridize to a second oligonucleotide functionalized on a second nanoparticle, and when present, a free oligonucleotide, having a sequence sufficiently complementary. In one aspect, methods are provided wherein each nanoparticle is functionalized with identical oligonucleotides, i.e., each oligonucleotide attached to the nanoparticle has the same length and the same sequence. In other aspects, each nanoparticle is functionalized with two or more oligonucleotides which are not identical, i.e., at least one of the attached oligonucleotides differ from at least one other attached oligonucleotide in that it has a different length and/or a different sequence.

In one aspect, oligonucleotides are designed which are identical to, or sufficiently homologous to, double-strand or triple-strand polynucleotide complexes that exist in nature, thereby allowing identification of compounds that interact with a naturally-occurring complex. Accordingly, oligonucleotides are in general prepared with knowledge of the known sequences. Methods of making oligonucleotides of a predetermined sequence are well-known. See, for example, Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are contemplated for both oligoribonucleotides and oligodeoxyribonucleotides (the well-known methods of synthesizing DNA are also useful for synthesizing RNA). Oligoribonucleotides and oligodeoxyribonucleotides can also be prepared enzymatically.

Alternatively, oligonucleotides are selected from a library. Preparation of libraries of this type is well know in the art. See, for example, Oligonucleotide libraries: United States Patent Application 20050214782, published Sep. 29, 2005.

### I. Oligonucleotide Length

The term “oligonucleotide” or “polynucleotide” as used herein includes modified forms as discussed herein as well as those otherwise known in the art which are used to regulate gene expression. Likewise, the term “nucleotides” as used herein is interchangeable with modified forms as discussed herein and otherwise known in the art. In certain instances, the art uses the term “nucleobase” which embraces naturally-occurring nucleotides as well as modifications of nucleotides that can be polymerized. Herein, the terms “nucleotides” and “nucleobases” are used interchangeably to embrace the same scope unless otherwise noted.

Nanoparticles for use in the methods provided are functionalized with an oligonucleotide, or modified form thereof, which is from about 5 to about 100 nucleotides in length. Methods are also contemplated wherein the oligonucleotide is about 5 to about 90 nucleotides in length, about 5 to about 80 nucleotides in length, about 5 to about 70 nucleotides in length, about 5 to about 60 nucleotides in length, about 5 to about 50 nucleotides in length about 5 to about 45 nucleotides in length, about 5 to about 40 nucleotides in length, about 5 to about 35 nucleotides in length, about 5 to about 30 nucleotides in length, about 5 to about 25 nucleotides in length, about 5 to about 20 nucleotides in length, about 5 to about 15 nucleotides in length, about 5 to about 10 nucleotides in length, and all oligonucleotides intermediate in length of the sizes specifically disclosed to the extent that the oligonucleotide is able to achieve the desired result. Accordingly, oligonucleotides of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, and 100 nucleotides in length are contemplated.

In still other aspects, oligonucleotides comprise from about 8 to about 80 nucleotides (i.e. from about 8 to about 80 linked nucleosides). One of ordinary skill in the art will appreciate that methods utilize compounds of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleotide in length.

### J. Oligonucleotide Complementarity

“Hybridization,” which is used interchangeably with the term “complex formation” herein, means an interaction between two or three strands of nucleic acids by hydrogen bonds in accordance with the rules of Watson-Crick DNA complementarity, Hoogstein binding, or other sequence-specific binding known in the art. Hybridization can be performed under different stringency conditions known in the art. Under appropriate stringency conditions, hybridization between the two complementary strands could reach about 60% or above, about 70% or above, about 80% or above, about 90% or above, about 95% or above, about 96% or above, about 97% or above, about 98% or above, or about 99% or above in the reactions.

In various aspects, the methods include use of two or three oligonucleotides which are 100% complementary to each other, i.e., a perfect match, while in other aspects, the individual oligonucleotides are at least (meaning greater than or equal to) about 95% complementary to each over the all or part of length of each oligonucleotide, at least about 90%, at least about 85%, at least about 80%, at least about 75%, at least about 70%, at least about 65%, at least about 60%, at least about 55%, at least about 50%, at least about 45%, at least about 40%, at least about 35%, at least about 30%, at least about 25%, at least about 20% complementary to each other.

It is understood in the art that the sequence of the oligonucleotide used in the methods need not be 100% complementary to each other to be specifically hybridizable. Moreover, oligonucleotide may hybridize to each other over one or more segments such that intervening or adjacent segments are not involved in the hybridization event (e.g., a loop structure or hairpin structure). Percent complementarity between any given oligonucleotide can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

### K. Oligonucleotide Attachment

Oligonucleotides contemplated for use in the methods include those bound to the nanoparticle through any means. Regardless of the means by which the oligonucleotide is attached to the nanoparticle, attachment in various aspects is effected through a 5′ linkage, a 3′ linkage, some type of internal linkage, or any combination of these attachments.

In one aspect, the nanoparticles, the oligonucleotides or both are functionalized in order to attach the oligonucleotides to the nanoparticles. Methods to functionalize nanoparticles and oligonucleotides are known in the art. For instance, oligonucleotides functionalized with alkanethiols at their 3′-termini or 5′-termini readily attach to gold nanoparticles. See Whitesides, Proceedings of the Robert A. Welch Foundation 39th Conference On Chemical Research Nanophase Chemistry, Houston, Tex., pages 109-121 (1995). See also, Mucic et al. Chem. Commun. 555-557 (1996) which describes a method of attaching 3′ thiol DNA to flat gold surfaces. The alkanethiol method can also be used to attach oligonucleotides to other metal, semiconductor and magnetic colloids and to the other types of nanoparticles described herein. Other functional groups for attaching oligonucleotides to solid surfaces include phosphorothioate groups (see, for example, U.S. Pat. No. 5,472,881 for the binding of oligonucleotide-phosphorothioates to gold surfaces), substituted alkylsiloxanes (see, for example, Burwell, Chemical Technology, 4, 370-377 (1974) and Matteucci and Caruthers, J. Am. Chem. Soc., 103, 3185-3191 (1981) for binding of oligonucleotides to silica and glass surfaces, and Grabar et al., Anal. Chem., 67, 735-743 for binding of aminoalkylsiloxanes and for similar binding of mercaptoaklylsiloxanes). Oligonucleotides with a 5′ thionucleoside or a 3′ thionucleoside may also be used for attaching oligonucleotides to solid surfaces. The following references describe other methods which may be employed to attached oligonucleotides to nanoparticles: Nuzzo et al., J. Am. Chem. Soc., 109, 2358 (1987) (disulfides on gold); Allara and Nuzzo, Langmuir, 1, 45 (1985) (carboxylic acids on aluminum); Allara and Tompkins, J. Colloid Interface Sci., 49, 410-421 (1974) (carboxylic acids on copper); Iler, The Chemistry Of Silica, Chapter 6, (Wiley 1979) (carboxylic acids on silica); Timmons and Zisman, J. Phys. Chem., 69, 984-990 (1965) (carboxylic acids on platinum); Soriaga and Hubbard, J. Am. Chem. Soc., 104, 3937 (1982) (aromatic ring compounds on platinum); Hubbard, Acc. Chem. Res., 13, 177 (1980) (sulfolanes, sulfoxides and other functionalized solvents on platinum); Hickman et al., J. Am. Chem. Soc., 111, 7271 (1989) (isonitriles on platinum); Maoz and Sagiv, Langmuir, 3, 1045 (1987) (silanes on silica); Maoz and Sagiv, Langmuir, 3, 1034 (1987) (silanes on silica); Wasserman et al., Langmuir, 5, 1074 (1989) (silanes on silica); Eltekova and Eltekov, Langmuir, 3, 951 (1987) (aromatic carboxylic acids, aldehydes, alcohols and methoxy groups on titanium dioxide and silica); Lec et al., J. Phys. Chem., 92, 2597 (1988) (rigid phosphates on metals).

U.S. patent application Ser. Nos. 09/760,500 and 09/820,279 and international application nos. PCT/US01/01190 and PCT/US01/10071 describe oligonucleotides functionalized with a cyclic disulfide. The cyclic disulfides in certain aspects have 5 or 6 atoms in their rings, including the two sulfur atoms. Suitable cyclic disulfides are available commercially or are synthesized by known procedures. Functionalization with the reduced forms of the cyclic disulfides is also contemplated.

In certain aspects wherein cyclic disulfide functionalization, oligonucleotides are attached to a nanoparticle through one or more linkers. In one embodiment, the linker comprises a hydrocarbon moiety attached to a cyclic disulfide. Suitable hydrocarbons are available commercially, and are attached to the cyclic disulfides. The hydrocarbon moiety is, in one aspect, a steroid residue. Oligonucleotide-nanoparticle conjugates prepared using linkers comprising a steroid residue attached to a cyclic disulfide are more stable to thiols compared to conjugates prepared using alkanethiols or acyclic disulfides as the linker, and in certain instances, the oligonucleotide-nanoparticle conjugates have been found to be 300 times more stable. In certain embodiments, the two sulfur atoms of the cyclic disulfide are close enough together so that both of the sulfur atoms attach simultaneously to the nanoparticle. In other aspects, the two sulfur atoms are adjacent each other. In aspects where utilized, the hydrocarbon moiety is large enough to present a hydrophobic surface screening the surfaces of the nanoparticle.

In other aspects, a method for attaching oligonucleotides onto a surface is based on an aging process described in U.S. application Ser. No. 09/344,667, filed Jun. 25, 1999; Ser. No. 09/603,830, filed Jun. 26, 2000; Ser. No. 09/760,500, filed Jan. 12, 2001; Ser. No. 09/820,279, filed Mar. 28, 2001; Ser. No. 09/927,777, filed Aug. 10, 2001; and in International application nos. PCT/US97/12783, filed Jul. 21, 1997; PCT/US00/17507, filed Jun. 26, 2000; PCT/US01/01190, filed Jan. 12, 2001; PCT/US01/10071, filed Mar. 28, 2001, the disclosures which are incorporated by reference in their entirety. The aging process provides nanoparticle-oligonucleotide conjugates with enhanced stability and selectivity. The process comprises providing oligonucleotides, in one aspect, having covalently bound thereto a moiety comprising a functional group which can bind to the nanoparticles. The moieties and functional groups are those that allow for binding (i.e., by chemisorption or covalent bonding) of the oligonucleotides to nanoparticles. For example, oligonucleotides having an alkanethiol, an alkanedisulfide or a cyclic disulfide covalently bound to their 5′ or 3′ ends bind the oligonucleotides to a variety of nanoparticles, including gold nanoparticles.

Conjugates produced by use of the “aging” step have been found to be considerably more stable than those produced without the “aging” step. Increased density of the oligonucleotides on the surfaces of the nanoparticles is achieved by the “aging” step. The surface density achieved by the “aging” step will depend on the size and type of nanoparticles and on the length, sequence and concentration of the oligonucleotides. A surface density adequate to make the nanoparticles stable and the conditions necessary to obtain it for a desired combination of nanoparticles and oligonucleotides can be determined empirically. Generally, a surface density of at least 10 picomoles/cm² will be adequate to provide stable nanoparticle-oligonucleotide conjugates. In certain aspects, the surface density is at least 15 picomoles/cm². Since the ability of the oligonucleotides of the conjugates to hybridize with nucleic acid and oligonucleotide targets can be diminished if the surface density is too great, the surface density is, in one aspect, no greater than about 35-40 picomoles/cm². Regardless, various oligonucleotide densities are contemplated as disclosed herein.

An “aging” step is incorporated into production of functionalized nanoparticles following an initial binding or oligonucleotides to a nanoparticle. In brief, the oligonucleotides are contacted with the nanoparticles in water for a time sufficient to allow at least some of the oligonucleotides to bind to the nanoparticles by means of the functional groups. Such times can be determined empirically. In one aspect, a time of about 12-24 hours is contemplated. Other suitable conditions for binding of the oligonucleotides can also be determined empirically. For example, a concentration of about 10-20 nM nanoparticles and incubation at room temperature is contemplated.

Next, at least one salt is added to the water to form a salt solution. The salt is any water-soluble salt, including, for example and without limitation, sodium chloride, magnesium chloride, potassium chloride, ammonium chloride, sodium acetate, ammonium acetate, a combination of two or more of these salts, or one of these salts in phosphate buffer. The salt is added as a concentrated solution, or in the alternative as a solid. In various embodiments, the salt is added all at one time or the salt is added gradually over time. By “gradually over time” is meant that the salt is added in at least two portions at intervals spaced apart by a period of time. Suitable time intervals can be determined empirically.

The ionic strength of the salt solution must be sufficient to overcome at least partially the electrostatic repulsion of the oligonucleotides from each other and, either the electrostatic attraction of the negatively-charged oligonucleotides for positively-charged nanoparticles, or the electrostatic repulsion of the negatively-charged oligonucleotides from negatively-charged nanoparticles. Gradually reducing the electrostatic attraction and repulsion by adding the salt gradually over time gives the highest surface density of oligonucleotides on the nanoparticles. Suitable ionic strengths can be determined empirically for each salt or combination of salts. In one aspect, a final concentration of sodium chloride of from about 0.1 M to about 1.0 M in phosphate buffer is utilized, with the concentration of sodium chloride being increased gradually over time.

After adding the salt, the oligonucleotides and nanoparticles are incubated in the salt solution for a period of time to allow additional oligonucleotides to bind to the nanoparticles to produce the stable nanoparticle-oligonucleotide conjugates. As will be described in detail below, an increased surface density of the oligonucleotides on the nanoparticles stabilizes the conjugates. The time of this incubation can be determined empirically. By way of example, in one aspect a total incubation time of about 24-48, wherein the salt concentration is increased gradually over this total time, is contemplated. This second period of incubation in the salt solution is referred to herein as the “aging” step. Other suitable conditions for this “aging” step can also be determined empirically. By way of example, an aging step is carried out with incubation at room temperature and pH 7.0.

The conjugates produced by use of the “aging” are in general more stable than those produced without the “aging” step. As noted above, this increased stability is due to the increased density of the oligonucleotides on the surfaces of the nanoparticles which is achieved by the “aging” step. The surface density achieved by the “aging” step will depend on the size and type of nanoparticles and on the length, sequence and concentration of the oligonucleotides.

As used in this context, “stable” means that, for a period of at least six months after the conjugates are made, a majority of the oligonucleotides remain attached to the nanoparticles and the oligonucleotides are able to hybridize with nucleic acid and oligonucleotide targets under standard conditions encountered in methods of detecting nucleic acid and methods of nanofabrication.

An alternative “fast salt aging” process produced particles with comparable DNA densities and stability. By performing the salt additions in the presence of a surfactant, for example approximately 0.01% sodium dodecylsulfate (SDS), Tween, or polyethylele glycol (PEG), the salt aging process can be performed in about an hour.

### L. Oligonucleotide Density

Method are provided wherein the oligonucleotide is bound to the nanoparticle at a surface density of at least 10 pmol/cm², at least 15 pmol/cm², at least 20 pmol/cm², at least 25 pmol/cm², at least 30 pmol/cm², at least 35 pmol/cm², at least 40 pmol/cm², at least 45 pmol/cm², at least 50 pmol/cm², or 50 pmol/cm² or more.

In one aspect, methods are provided wherein the packing density of the oligonucleotides on the surface of the nanoparticle is sufficient to result in cooperative behavior between nanoparticles and between polynucleotide strands on a single nanoparticle. In another aspect, the cooperative behavior between the nanoparticles increases the resistance of the oligonucleotide to degradation.

### M. Oligonucleotide Copies—Same/Different Sequences

The term “oligonucleotide” or “polynucleotide” includes those wherein a single sequence is attached to a nanoparticle, or multiple copies of the single sequence are attached. For example, in various aspects, an oligonucleotide is present in multiple copies in tandem, for example, two, three, four, five, six, seven eight, nine, ten or more tandem repeats.

Alternatively, the nanoparticle is functionalized to include at least two oligonucleotides having different sequences. As above, the different oligonucleotide sequences are in various aspects arranged in tandem and/or in multiple copies. Alternatively, the oligonucleotides having different sequences are attached directly to the nanoparticle. In methods wherein oligonucleotides having different sequences are attached to the nanoparticle, aspects of the methods include those wherein the different oligonucleotide sequences hybridize to different regions on the same polynucleotide.

The oligonucleotides on the nanoparticles may all have the same sequence or may have different sequences that hybridize with different portions of the polynucleotide attached to another nanoparticle. When oligonucleotides having different sequences are used, each nanoparticle may have all of the different oligonucleotides attached to it or the different oligonucleotides are attached to different nanoparticles. Alternatively, the oligonucleotides on each of the nanoparticles may have a plurality of different sequences, at least one of which must hybridize with a portion of the polynucleotide on a second nanoparticle.

### N. Spacers

In certain aspects, functionalized nanoparticles are contemplated which include those wherein an oligonucleotide is attached to the nanoparticle through a spacer. “Spacer” as used herein means a moiety that does not participate in modulating gene expression per se but which serves to increase distance between the nanoparticle and the functional oligonucleotide, or to increase distance between individual oligonucleotides when attached to the nanoparticle in multiple copies. Thus, spacers are contemplated being located between individual oligonucleotide in tandem, whether the oligonucleotides have the same sequence or have different sequences. In one aspect, the spacer when present is an organic moiety. In another aspect, the spacer is a polymer, including but not limited to a water-soluble polymer, a nucleic acid, a polypeptide, an oligosaccharide, a carbohydrate, a lipid, or combinations thereof.

In certain aspects, the spacer has a moiety covalently bound to it, the moiety comprising a functional group which can bind to the nanoparticles. These are the same moieties and functional groups as described above. As a result of the binding of the spacer to the nanoparticles, the oligonucleotide is spaced away from the surface of the nanoparticles and is more accessible for hybridization with its target. In instances wherein the spacer is a polynucleotide, the length of the spacer in various embodiments at least about 10 nucleotides, 10-30 nucleotides, or even greater than 30 nucleotides. The spacer may have any sequence which does not interfere with the ability of the oligonucleotides to become bound to the nanoparticles. The spacers should not have sequences complementary to each other or to that of the oligonucleotides. In certain aspects, the bases of the polynucleotide spacer are all adenines, all thymines, all cytidines, all guanines, all uracils, or all some other modified base.

In another embodiment, a non-nucleotide linker of the invention comprises a basic nucleotide, polyether, polyamine, polyamide, peptide, carbohydrate, lipid, polyhydrocarbon, or other polymeric compounds. Specific examples include those described by Seela and Kaiser, Nucleic Acids Res. 1990, 18:6353 and Nucleic Acids Res. 1987, 15:3113; Cload and Schepartz, J. Am. Chem. Soc. 1991, 113:6324; Richardson and Schepartz, J. Am. Chem. Soc. 1991, 113:5109; Ma et al., Nucleic Acids Res. 1993, 21:2585 and Biochemistry 1993, 32:1751; Durand et al., Nucleic Acids Res. 1990, 18:6353; McCurdy et al., Nucleosides & Nucleotides 1991, 10:287; Jschke et al., Tetrahedron Lett. 1993, 34:301; Ono et al., Biochemistry 1991, 30:9914; Arnold et al., International Publication No. WO 89/02439; Usman et al., International Publication No. WO 95/06731; Dudycz et al., International Publication No. WO 95/11910 and Ferentz and Verdine, J. Am. Chem. Soc. 1991, 113:4000, the disclosures of which are all incorporated by reference herein. A “non-nucleotide” further means any group or compound that can be incorporated into a nucleic acid chain in the place of one or more nucleotide units, including either sugar and/or phosphate substitutions, and allows the remaining bases to exhibit their enzymatic activity. The group or compound can be abasic in that it does not contain a commonly recognized nucleotide base, such as adenosine, guanine, cytosine, uracil or thymine, for example at the C1 position of the sugar.

In various aspects, linkers contemplated include linear polymers (e.g., polyethylene glycol, polylysine, dextran, etc.), branched-chain polymers (see, for example, U.S. Pat. No. 4,289,872 to Denkenwalter et al., issued Sep. 15, 1981; 5,229,490 to Tam, issued Jul. 20, 1993; WO 93/21259 by Frechet et al., published 28 Oct. 1993); lipids; cholesterol groups (such as a steroid); or carbohydrates or oligosaccharides. Other linkers include one or more water soluble polymer attachments such as polyoxyethylene glycol, or polypropylene glycol as described U.S. Pat. Nos. 4,640,835, 4,496,689, 4,301,144, 4,670,417, 4,791,192 and 4,179,337. Other useful polymers as linkers known in the art include monomethoxy-polyethylene glycol, dextran, cellulose, or other carbohydrate based polymers, poly-(N-vinyl pyrrolidone)-polyethylene glycol, propylene glycol homopolymers, a polypropylene oxide/ethylene oxide co-polymer, polyoxyethylated polyols (e.g., glycerol) and polyvinyl alcohol, as well as mixtures of these polymers.

In still other aspects, oligonucleotide such as poly-A or hydrophilic or amphiphilic polymers are contemplated as linkers, including, for example, amphiphiles (including oligonucleotides).

### O. Types of Oligonucleotides, Including Modified Forms

In various aspects, methods include oligonucleotides which are DNA oligonucleotides, RNA oligonucleotides, or combinations of the two types. Modified forms of oligonucleotides are also contemplated which include those having at least one modified internucleotide linkage. In one embodiment, the oligonucleotide is all or in part a peptide nucleic acid. Other modified internucleoside linkages include at least one phosphorothioate linkage. Still other modified oligonucleotides include those comprising one or more universal bases. “Universal base” refers to molecules capable of substituting for binding to any one of A, C, G, T and U in nucleic acids by forming hydrogen bonds without significant structure destabilization. The oligonucleotide incorporated with the universal base analogues is able to function as a probe in hybridization, as a primer in PCR and DNA sequencing. Examples of universal bases include but are not limited to 5′-nitroindole-2′-deoxyriboside, 3-nitropyrrole, inosine and pypoxanthine.

1. Modified Internucleoside Linkages

Specific examples of oligonucleotides include those containing modified backbones or non-natural internucleoside linkages. Oligonucleotides having modified backbones include those that retain a phosphorus atom in the backbone and those that do not have a phosphorus atom in the backbone. Modified oligonucleotides that do not have a phosphorus atom in their internucleoside backbone are considered to be within the meaning of “oligonucleotide.”

Modified oligonucleotide backbones containing a phosphorus atom include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Also contemplated are oligonucleotides having inverted polarity comprising a single 3′ to 3′ linkage at the 3′-most internucleotide linkage, i.e. a single inverted nucleoside residue which may be abasic (the nucleotide is missing or has a hydroxyl group in place thereof). Salts, mixed salts and free acid forms are also contemplated. Representative United States patents that teach the preparation of the above phosphorus-containing linkages include, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050, the disclosures of which are incorporated by reference herein.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages; siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts. See, for example, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439, the disclosures of which are incorporated herein by reference in their entireties.

2. Modified Sugar and Internucleoside Linkages

In still other embodiments, oligonucleotide mimetics wherein both one or more sugar and/or one or more internucleotide linkage of the nucleotide units are replaced with “non-naturally occurring” groups. In one aspect, this embodiment contemplates a peptide nucleic acid (PNA). In PNA compounds, the sugar-backbone of an oligonucleotide is replaced with an amide containing backbone. See, for example U.S. Pat. Nos. 5,539,082; 5,714,331; and 5,719,262, and Nielsen et al., Science, 1991, 254, 1497-1500, the disclosures of which are herein incorporated by reference.

In still other embodiments, oligonucleotides are provided with phosphorothioate backbones and oligonucleosides with heteroatom backbones, and including —CH₂—NH—O—CH₂—, —CH₂—N(CH₃)—O—CH₂—, —CH₂—O—N(CH₃)—CH₂—, —CH₂—N(CH₃)—N(CH₃)—CH₂— and —O—N(CH₃)—CH₂—CH₂— described in U.S. Pat. Nos. 5,489,677, and 5,602,240. Also contemplated are oligonucleotides with morpholino backbone structures described in U.S. Pat. No. 5,034,506.

In various forms, the linkage between two successive monomers in the oligo consists of 2 to 4, desirably 3, groups/atoms selected from —CH₂—, —O—, —S—, —NR^{H}—, >C═O, >C═NR^{H}, >C═S, —Si(R″)₂—, —SO—, —S(O)₂—, —P(O)₂—, —PO(BH₃)—, —P(O,S)—, —P(S)₂—, —PO(R″)—, —PO(OCH₃)—, and —PO(NHR^{H})—, where RH is selected from hydrogen and C₁₋₄-alkyl, and R″ is selected from C₁₋₆-alkyl and phenyl. Illustrative examples of such linkages are —CH₂—CH₂—CH₂—, —CH₂—CO—CH₂—, —CH₂—CHOH—CH₂—, —O—CH₂—O—, —O—CH₂—CH₂—, —O—CH₂—CH=(including R⁵ when used as a linkage to a succeeding monomer), —CH₂—CH₂—O—, —NR^{H}—CH₂—CH₂—, —CH₂—CH₂—NR^{H}, —CH₂—NR^{H}—CH₂—, —O—CH₂—CH₂—NR^{H}, —NR^{H}—CO—O—, —NR^{H}—CO—NR^{H}—, —NR^{H}—CS—NR^{H}—, —NR^{H} —C(═NR^{H})—NR^{H}—, —NR^{H}—CO—CH₂—NR^{H}—O—CO—O—, —O—CO—CH₂—O—, —O—CH₂—CO—O—, —CH₂—CO—NR^{H}—, —O—CO—NR^{H}—, —NR^{H}—CO—CH₂—, —O—CH₂—CO—NR^{H}—, —O—CH₂—CH₂—NR^{H}—, —CH═N—O—, —CH₂—NR^{H}—, —CH₂—O—N=(including R⁵ when used as a linkage to a succeeding monomer), —CH₂—O—NR^{H}—, —CO—NR^{H}—CH₂—, —CH₂—NR^{H}—O—, —CH₂—NR^{H}—CO—, O—NR^{H}—CH₂—, O—NR^{H}, —O—CH₂—S—, —S—CH₂—O—, —CH₂—CH₂—S—, —O—CH₂—CH₂—S—, —S—CH₂—CH=(including R⁵ when used as a linkage to a succeeding monomer), —S—CH₂—CH₂—, —S—CH₂—CH₂—O—, —S—CH₂—CH₂—S—, —CH₂—S—CH₂—, —CH₂—SO—CH₂—, —CH₂—SO₂—CH₂—, —O—SO—O—, —O—S(O)₂—O—, —O—S(O)₂—CH₂—, —O—S(O)₂—NR^{H}—, —NR^{H}—S(O)₂—CH₂—; —O—S(O)₂—CH₂—, —O—P(O)₂—, —O—P(O,S)—O—, —O—P(S)₂—O—, —S—P(O)₂—O—, —S—P(O,S)—O—, —S—P(S)₂—O—, —O—P(O)₂—S—, —O—P(O,S)—S—, —O—P(S)₂—S—, —S—P(O)₂—S—, —S—P(O,S)—S—, —S—P(S)₂—S—, —O—PO(R″)—O—, —O—PO(OCH₃)—O—, —PO(O CH₂CH₃)—O—, —O—PO(O CH₂CH₂S—R)—O—, —O—PO(BH₃)—O—, —O—PO(NHR^{N})—O—, O—P(O)₂—NR^{H} H—, —NR^{H}—P(O)₂—O—, —O—P(O,NR^{H})—O—, —CH₂—P(O)₂—, —O—P(O)₂—CH₂—, and —O—Si(R″)₂—O—; among which —CH₂—CO—NR^{H}—, —CH₂—NR^{H}—O—, —S—CH₂—O—, —P(O)₂—O—O—P(—O,S)—O—, —O—P(S)₂—O—, —NR^{H} P(O)₂—O—, —O—P(O,NR^{H})—O—, —O—PO(R″)—O—, —O—PO(CH₃)—O—, and —O—PO(NHR^{N})—O—, where RH is selected form hydrogen and C₁₋₄-alkyl, and R″ is selected from C₁₋₆-alkyl and phenyl, are contemplated. Further illustrative examples are given in Mesmaeker et. al., Current Opinion in Structural Biology 1995, 5, 343-355 and Susan M. Freier and Karl-Heinz Altmann, Nucleic Acids Research, 1997, vol 25, pp 4429-4443.

Still other modified forms of oligonucleotides are described in detail in U.S. patent application NO. 20040219565, the disclosure of which is incorporated by reference herein in its entirety.

Modified oligonucleotides may also contain one or more substituted sugar moieties. In certain aspects, oligonucleotides comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Other embodiments include O[(CH₂)ₙO]ₘCH₃, O(CH₂)ₙOCH₃, O(CH₂)ₙNH₂, O(CH₂)ₙCH₃, O(CH₂)ₙONH₂, and O(CH₂)ₙON[(CH₂)ₙCH₃]₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH2, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In one aspect, a modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. Other modifications include 2′-dimethylaminooxyethoxy, i.e., a O(CH₂)₂ON(CH₃)₂ group, also known as 2′-DMAOE, as described in examples herein below, and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂, also described in examples herein below.

Still other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. In one aspect, a 2′-arabino modification is 2′-F. Similar modifications may also be made at other positions on the oligonucleotide, for example, at the 3′ position of the sugar on the 3′ terminal nucleotide or in 2′-5′ linked oligonucleotides and the 5′ position of 5′ terminal nucleotide. Oligonucleotides may also have sugar mimetics such as cyclobutyl moieties in place of the pentofuranosyl sugar. See, for example, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920, the disclosures of which are incorporated by reference in their entireties herein.

In one aspect, a modification of the sugar includes Locked Nucleic Acids (LNAs) in which the 2′-hydroxyl group is linked to the 3′ or 4′ carbon atom of the sugar ring, thereby forming a bicyclic sugar moiety. The linkage is in certain aspects is a methylene (—CH₂—)ₙ group bridging the 2′ oxygen atom and the 4′ carbon atom wherein n is 1 or 2. LNAs and preparation thereof are described in WO 98/39352 and WO 99/14226.

3. Natural and Modified Bases

Oligonucleotides may also include base modifications or substitutions. As used herein, “unmodified” or “natural” bases include the purine bases adenine (A) and guanine (G), and the pyrimidine bases thymine (T), cytosine (C) and uracil (U). Modified bases include other synthetic and natural bases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil(pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine(1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Further bases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing the binding affinity and include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

A “modified base” or other similar term refers to a composition which can pair with a natural base (e.g., adenine, guanine, cytosine, uracil, and/or thymine) and/or can pair with a non-naturally occurring base. In certain aspects, the modified base provides a Tₘ differential of 15, 12, 10, 8, 6, 4, or 2° C. or less. Exemplary modified bases are described in EP 1 072 679 and WO 97/12896.

By “nucleobase” is meant the naturally occurring nucleobases adenine (A), guanine (G), cytosine (C), thymine (T) and uracil (U) as well as non-naturally occurring nucleobases such as xanthine, diaminopurine, 8-oxo-N⁶-methyladenine, 7-deazaxanthine, 7-deazaguanine, N⁴,N⁴-ethanocytosin, N′,N′-ethano-2,6-diaminopu-rine, 5-methylcytosine (mC), 5-(C³—C⁶)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, Nucleic Acids Research, 1997, vol. 25, pp 4429-4443. The term “nucleobase” thus includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanglivi, in Antisense Research and Application, Ed. S. T. Crooke and B. Lebleu, CRC Press, 1993, in Englisch et al., Angewandte Chemie, International Edition, 1991, 30, 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). The term “nucleosidic base” or “base unit” is further intended to include compounds such as heterocyclic compounds that can serve like nucleobases including certain “universal bases” that are not nucleosidic bases in the most classical sense but serve as nucleosidic bases. Especially mentioned as universal bases are 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

### Example 1

This method utilizes the aggregation-induced, red-to-blue color change associated with 13 nm gold (Au) nanoparticles. The assay consists of two sets of gold nanoparticles NP-1 and NP-2, and a free strand of DNA, DNA-3. NP-1 and NP-2 are functionalized with either 3′ or 5′ pyrimidine rich thiol-modified oligonucleotide strands which are noncomplementary and do not interact. DNA-3 is complementary to NP-1 with a two base dangling end to prevent noncross-linked NP-1 aggregation. When NP-1 and DNA-3 are combined, they form nonaggregate linking duplexes on the nanoparticle surface. NP-2 DNA has the proper sequence to form a triplex with the Initial NP-1/DNA-3 duplex, but due to the low stability of the triplex structure, aggregation does not form at room temperature. However, introduction of a strong triplex binding agent, benzo[e]pyridoindole (BePl), stabilizes triplex formation through Hoogsteen-type Py-PaPy triplet base hydrogen bonds and induces nanoparticle aggregation resulting in a concomitant red-to-blue color change due to a red-shifting and dampening of the nanoparticle plasmon resonance (Scheme 1). Introduction of a duplex binder does not stabilize the triplex structure and no aggregation is seen.

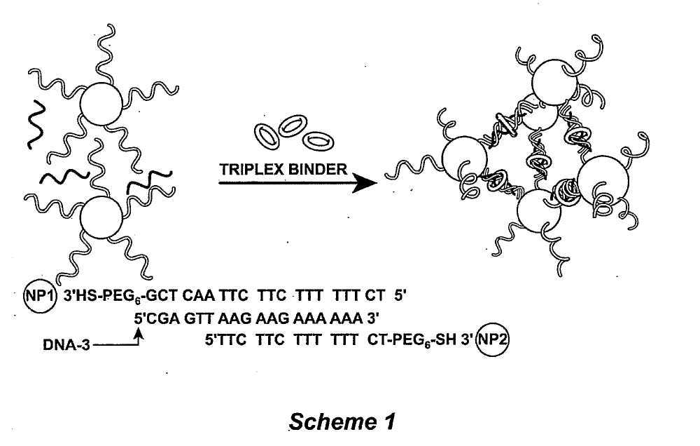

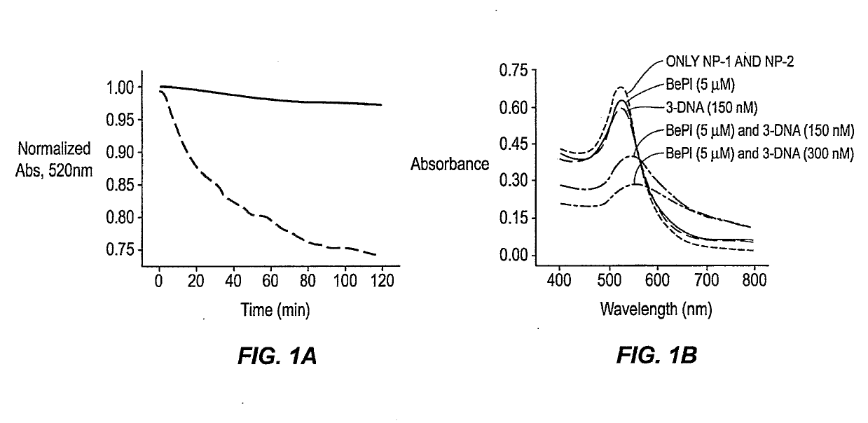

This assay is prepared by combining NP-1 and NP-2 (1.5 nM each) in a 1:1 molar ratio in 10 mM PBS buffer (pH=7.0, NaCl 0.5 M) with DNA-3 (150 nM) and BePl (5 μM). Kinetic analysis indicates that triplex formation and subsequent aggregation is dependent an intercalator and DNA-3 concentration (FIG. 1). In the absence of triplex binder, due to instability at room temperature, the triplex structure does not form. As a result, nanoparticle aggregation does not occur as observed by very little change in the surface plasmon at 520 nm and a constant red color. However in the presence of a strong triplex binder, BePI the triplex structure is stabilized and nanoparticle aggregation occurs. This stabilization results in a dramatic decrease in absorbance at 520 nm accompanied by a red-to-blue color change. Note that in the absence of DNA-3, NP-1 and NP-2 cannot form aggregates even in the presence of a strong triplex binder (FIG. 1B). These results demonstrate that nanoparticle aggregation is dependent on the presence of both DNA-3 and a triplex stabilizing binder.

### Example 2

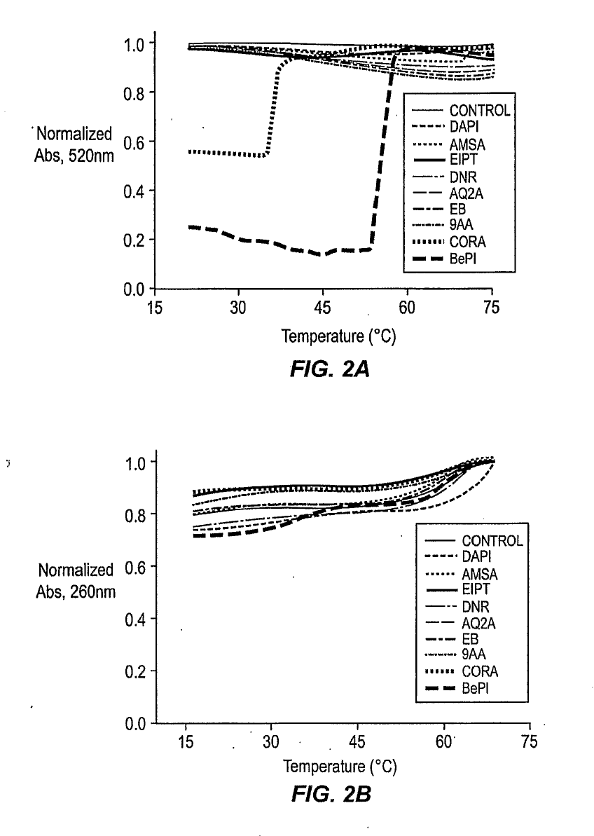

To further investigate the importance of the triplex structure and rule out aggregation due to duplex formation between the nanoparticles, the assay as set out in Example I was performed in the presence of seven additional duplex binders 4′,6-diamidino-2-phenylindole (DAPI), ellipticine (EIPT), amsacrine (AMSA), daunorubicin (DNR), anthraquinone-2-carboxylic acid (AQ2A), ethidium bromide (EB), and 9-aminoaclidine (9-AA)(5 μM). Unlike with BePI, no nanoparticle aggregation is seen in the presence of the duplex binders (FIG. 2A). The results show that only BePl, a strong triplex binder, can induce aggregation compared to the six other duplex binders.

### Example 3

To compare the results from the previous examples with more traditional screening techniques, all eight intercalators were screened, plus a control, by monitoring UV absorbance melting curves (260 nm) of unmodified DNA-1, DNA-2, and DNA-3 (1 μM each), with each intercalator (10 μM), (DNA-1; 3′ TTCTTCTTTTTT CT-5′. DNA-2: 5′-TTCTTCTTTTTTCT-3′, DNA-3: 5′-AAGAAGAAAAAA-3′). (FIG. 2B). The melting experiment performed in the presence of BePl has two melting transitions. The first Tₘ at 34.8° C. is associated with the denaturation of the triplex structure and the second Tₘ at 61.4° C. is representative of the corresponding duplex. None of the seven duplex binders or the control showed this second melting transition. These results confirmed that of the DNA binders used here, only BePl is a strong triplex intercalator.

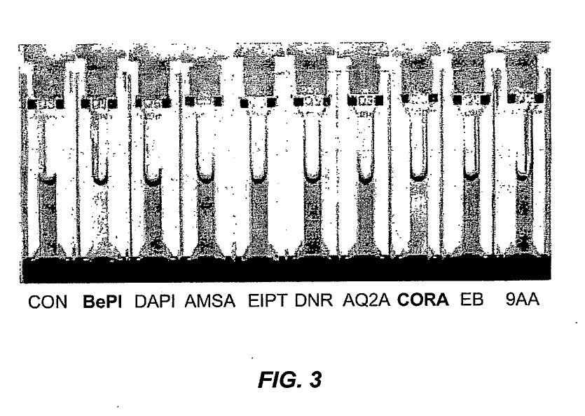

In general, assay methods that can detect drug candidates by the naked eye, without resorting to any instrumentation, are convenient, and for this reason, an assay that could screen for triplex binders would be of great interest. At present, there are no assays that provide this capability. The use of DNA functionalized Au nanoparticles for this purpose is demonstrated in FIG. 3. The mixtures of NP-1, NP-2 and DNA-3 containing the control and duplex intercalators remain red in color. Only the mixture containing the strong triplex binder, BePI, turns blue/purple in color. This result shows discrimination between triplex stabilizing sold and nonstabilizing binders by an easily identifiable color change. This result is consistent with the control experiments involving serial analysis of each DNA binder with nanoparticle-free triplex DNA.

### Example 4

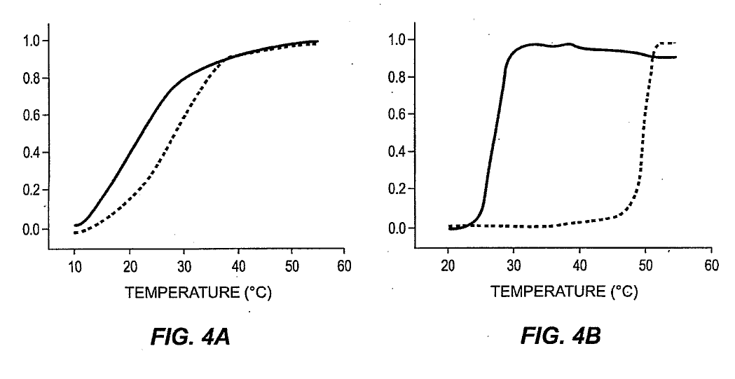

This method also utilizes the aggregation-induced, red-to-blue color change associated with 13 nm Au nanoparticles. Gold nanoparticles are functionalized with one of two complementary thiol-modified oligonucleotide strands and are denoted NP-1 and NP-2. When NP-1 and NP-2 are combined, they form aggregates through a reversible DNA hybridization process. This process results in a red-to-blue color change due to a red-shifting and dampening of the nanoparticle plasmon resonance. Increasing the temperature above the melting temperature of the DNA reverses the process, and the particles dissociate with a concomitant blue-to-red color change. The melting transition occurs over a very narrow temperature range with the first derivative of the transition exhibiting a FWHM of 1-2° C. This is significantly more narrow than the transition associated with nanoparticle-free duplex of identical length and sequence (FWHM of ˜10-12° C.), and is characteristic of nanoparticle and polymer probes heavily functionalized with oligonucleotide strands. When NP-1 and NP-2 are combined in presence of any of the known DNA intercalators in Table 1, duplexes form between the Au nanoparticles that are more stable than in the absence of intercalator (Scheme 2). This increased stability is reflected by an increase in melting temperature. Note that the shape and breadth of the transition is almost independent of the intercalator (FIG. 4). Therefore, by monitoring the blue-to-red color transition of hybridized oligonucleotide-modified Au nanoparticles in the presence of different compounds, the relative strength of intercalator binding can be determined with the naked-eye.

The assay is initiated by mixing NP-1 and NP-2 (1.5 nM each) in a 1:1 molar ratio in 10 mM PBS buffer (pH=7.0, NaCl 0.1 M. The melting temperatures of the nanoassemblies (NP-1 and NP-2) were then determined in the presence of 4′,6-diamidino-2-phenylindole (DAPI), ellipticine (EIPT), Amsacrine (AMSA), daunorubicin (DNR), anthraquinone-2-carboxylic acid (AQ2A), ethidium bromide (EB), and 9-aminoacridine (9-AA) (5 μM), respectively. UV melting profiles were measured by monitoring the absorbance at 520 nm at a scan rate of 0.5° C. min⁻¹. Melting temperatures were determined by taking the maxima of the first derivative of the melting curves. Control experiments were run by determining the melting temperatures of unmodified duplex DNA (DNA-1: 5′-TAACAATAA-3′, DNA-2: 5′-TTATTGTTA-3′) in the presence of all seven intercalators shown in Table 1.

TABLE 1
Melting temperatures (Tₘ) of nanoassemblies and control
duplexes in the presence of intercalators

| Compounds | Tₘ^{a} | Tₘ^{b} |
|---|---|---|
| Control | 26.4 | 19.1 |
| DAPI | 50.4 | 27.8 |
| DNR | 38.4 | 25.4 |
| EB | 37.8 | 21.8 |
| EIPT | 29.2 | 20.5 |
| AMSA | 27.2 | 19.5 |
| AQ2A | 28.5 | 19.4 |
| 9-AA | 30.9 | 19.3 |

The strength of binding between anticancer drugs and DNA generally correlates with the drug's biological activity and is reflected in an increased melting temperature for the DNA. In this way, we can screen for anticancer drug candidates by finding strong intercalators based on DNA melting temperature date. Because the absolute values of the DNA melting temperatures are dependent on many factors such as salt and DNA concentrations, only the trend of the melting temperature is critical. In comparing the nanoparticle melting data and the unmodified duplex control melting data (with and without intercalators), it can be seen that the trends in the melting temperatures are, for the most part, identical.

The nanoparticles also provide two additional advantages. The melting transition occurs over a very narrow temperature range, which allows for a more precise analysis of temperature change. In addition, the presence of the intercalator induces a more substantial temperature increase in the nanoparticle system as compared with the nanoparticle-free duplex system (Table 1), increasing our ability to easily identify strong intercalators. The dramatic increase in melting temperature of the nanoparticle aggregates in the presence of intercalator is due to a extremely high concentration ratio of intercalator to nanoparticle DNA (FIG. 4B, ratio 33:1) as compared the nanoparticle free case (FIG. 4A, ratio=5:2). This result is possible because the nanoparticles are much stronger light absorbers (at 260 nm or 520 nm) than the DNA, allowing one of skill in the art to work at very low nanoparticle probe concentration in the intercalator screening experiments.

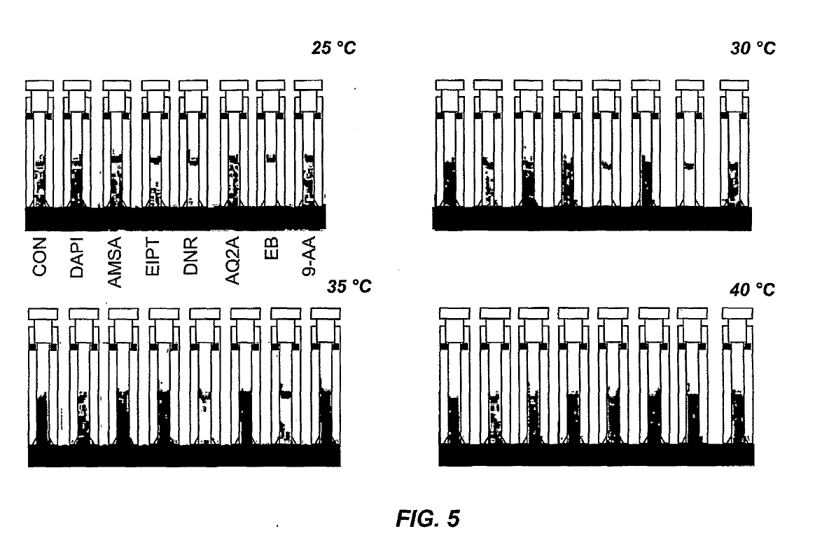

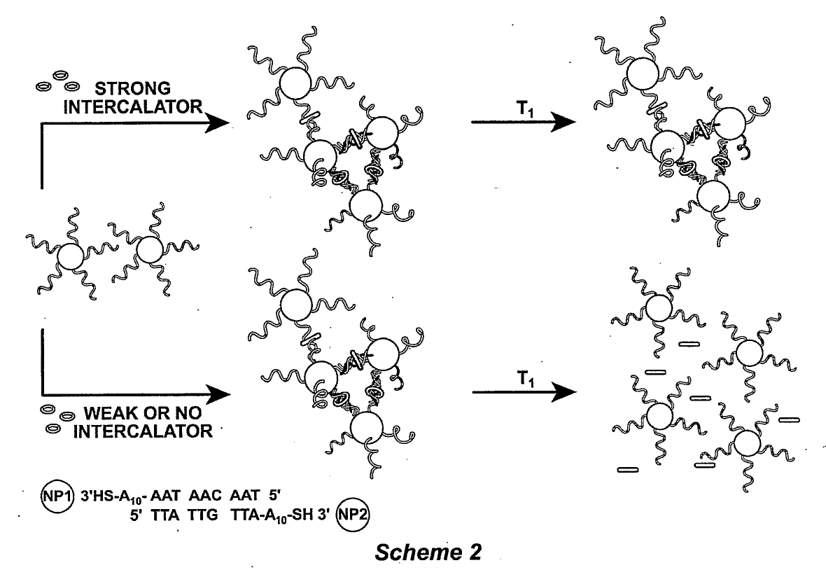

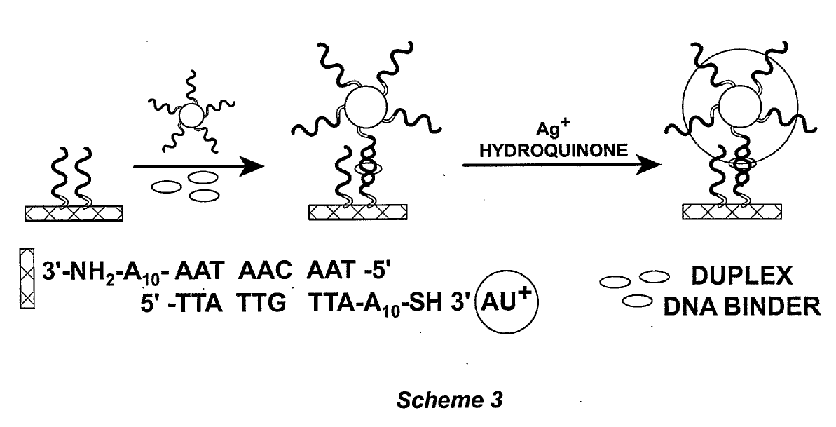

In general, assay methods that can detect drug candidates by the naked eye, without resorting to any instrumentation, are convenient, and for this reason, an assay that could seem for drug candidates based on DNA intercalation would be of great interest. The use of DNA functionalized Au nanoparticles for this purpose is demonstrated in FIG. 5. As the temperature increases, the color change from blue to red occurs at specific temperatures. At 25° C. all eight cells (one control and seven intercalators) appear light blue/purple in color. At 30° C., the nanoassemblies containing the control and weak intercalators turn red in color leaving only the nanoassemblies containing DAPI, DNR, and EB as blue/purple. Increasing the temperature to 40° C. causes all of the samples to turn red except the nanoassembly containing DAPI, a strong intercalator. This shows discrimination between weak, intermediate and strong intercalators by an easily identified color change. The trend of intercalator binding affinities for DNA was determined to be DAPI>DNR˜EB>other intercalators, which is consistent with the control experiments involving serial analysis of each intercalator with nanoparticle-free duplex DNA.

### Example 5

All DNAs used in this paper were purchased from Integrated DNA Technologies. All buffer solutions were prepared using reagents purchased from Sigma-Alrdich (St. Louis Mo.).

Au nanoparticles (13 nm diameter), prepared by citrate reduction of HAuCl4 were used. Au nanoparticle were functionalized by derivatizing aqueous Au colloid with thiol-modified oligonucleotides (final concentration of oligonucleotides ˜4 μM). After 12 hr the colloid solution was brought to 10 mM phosphate (NaH₂PO₄/Na₂HPO₄) buffer by adding 1.0 M pH 7 concentrated buffer. In the subsequent salt aging steps, colloids were brought to 0.05 M NaCl by dropwise addition of 2 M NaCl solution and allowed to stand for 6-8 h. This process was repeated to increase the salt concentration to 0.1, 0.2 and 0.3 M NaCl. To remove excess thiol-DNA, the solution was centrifuged using 1.5 mL Eppendorf tubes (Fisher Scientific) (30 min at 15 000 rpm). Following removal of the supernatant, the oily precipitate was suspended with distilled water. This process was repeated five times with a final resuspension in 0.5 M NaCl 10 mM PBS 0.001% NaN₃.

### Example 6

### Determine Melting Temperature of Control DNA (No Nanoparticles)

To determine the Tₘ of the duplex DNA in the presence of DNA binders, 2 μM duplex DNA and 5 μM DNA binder were combined in 0.3 M NaCl 10 mM PBS. To determine the Tₘ of the triplex DNA in the presence of DNA binders, 1 μM of each strand and 6 μM of each DNA binder were combined in 0.5 M NaCl 10 mM PBS. UV-vis melting curves were measured on a CARRY 500 spectrophotometer equipped with a circulating bath. The melting process was monitored by measuring the change in extinction at 260 nm. The solutions were stirred continuously with a magnetic stir bar as the solution temperature was increased at a rate of 0.5° C./min. The first derivative generated from the melting curves was used to determine the Tₘ.

### Preparation of Oligonucleotide Arrays

N-Hydroxysuccinimide-activated Codelink glass microarray slides (Amersham, G. E. Healthcare) were arrayed with amine-terminated oligonucleotides (5′-TAA CAA TAA-A10-NH2-3′, Integrated DNA Technologies, Inc.) according to the manufacturer's protocol. The oligonucleotides were printed in triplicate using a GME 418 robotic pin-and-ring microarrayer (Affymetrix).

### Scanometric Detection of Duplex and Triplex DNA Binding Molecules

For scanometric detection of duplex or triplex DNA binding molecules, Au nanoparticles (1 nM) and DNA binding molecules (5 μM) in 10 mM PBS buffer of varying NaCl concentrations were added to the microarray slide under hybridization wells (Nanosphere, Inc.). For detection of triplex binders, 150 nM DNA-3 was added. DNA hybridization proceeded for 30 min (for duplex DNA binders) or 1 h (for triplex DNA binders) in a humidity chamber. After hybridization, the slides were washed three times with 0.5 M NaNO₃ containing 0.02% Tween 20. The arrays were dried using a benchtop spinner. Silver enhancement solution (Nanosphere, Inc.) was added for Au-NP signal amplification (1 mL total volume/array, 1.5-min development time for duplex DNA binding molecules and 3 min for triplex binding molecules). The reaction was terminated by washing the slides with NANOpure® water and spin-drying. Light-scattering images from the silver-enhanced chips were recorded using the high-resolution Verigene ID system (Nanosphere, Inc.).

### Example 7

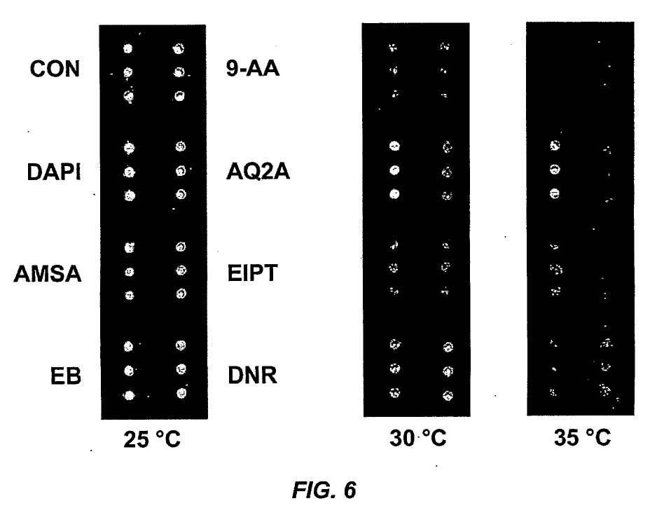

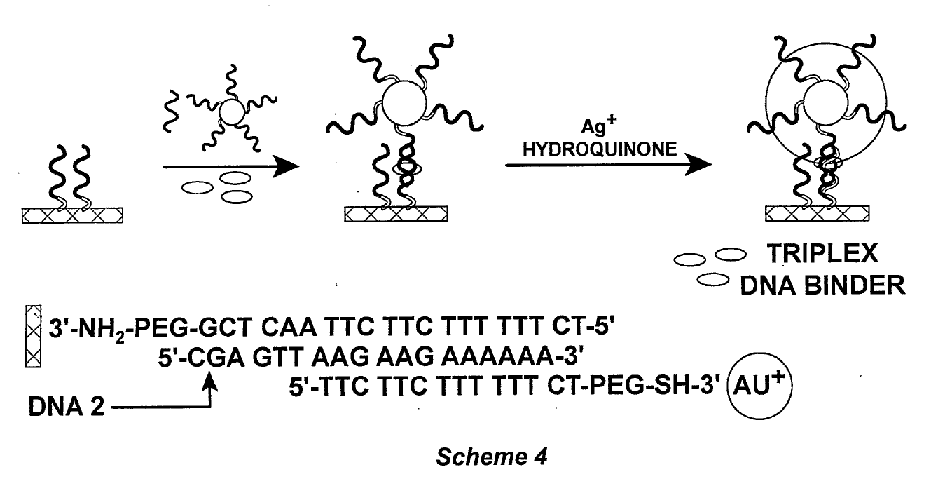

Capture strands of synthetic oligonucleotides are patterned on a glass chip using a microarrayer. The DNA chip is then exposed to DNA functionalized Au nanoparticles (complementary to the capture sequence) and a duplex DNA binder. The nanoparticle-bound DNA hybridizes to the capture strand and forms a duplex, simultaneously immobilizing the nanoparticle on the chip surface and providing a binding site for the duplex DNA binder, Scheme 3. The surface bound nanoparticles are exposed to a silver enhancement process. The presence of the Au nanoparticles catalyzes the reduction of silver, resulting in silver deposition around the nanoparticles. The light scattering from the silver enhancement is then visualized by a Verigene ID scanner. (FIG. 6)

The hybridization of single stranded DNA to form duplex DNA is a reversible process and the stability of the duplex is affected by several factors such as temperature and salt concentration. In addition, the presence of the duplex DNA binders increases the stability of the DNA duplex and is reflected by an increase in the duplex Tₘ. The strength of binding of the duplex DNA binder is represented by the magnitude of increased Tₘ. Therefore, by performing the nanoparticle hybridization process in the presence of different duplex DNA binders as a function of temperature, the relative binding strength of duplex DNA binders can be determined.

### Example 8

Analysis of seven known duplex DNA binders was performed to demonstrate the ability of this method to discriminate between strong, intermediate and weak binders. At 25° C., all seven duplex DNA binders (listed in Table 2) and a control showed positive signal after silver enhancement.

Increasing the temperature to 30° C. during the hybridization process eliminates signal from the control and the weak duplex DNA binders, leaving the intermediate and strong DNA binders: EB, DNR and DAPI. Further increasing the temperature eliminates the signal from the intermediate strength binders leaving only the strong duplex DNA binder, DAPI.

These results correlate well with control experiments which look at the Tₘ of solution-phase unmodified DNA in the presence of the seven duplex DNA binders, Table 2. The relative trend in binding strength determined from the control experiments is DAPI>DNR>EB>other intercalators. This is in agreement with the trend in relative binding strengths determined by the chip-based scanometric method.

TABLE 2
Melting temperatures of solution-phase unmodified DNA in the
presence of duplex DNA binders (2 uM duplex DNA, 5 uM DNA binder:
4′,6-diamidino-2-phenylindole (DAPI), ellipticine (EIPT),
Amsacrine (AMSA), daunorubicin (DNR), anthraquinone-2-carboxylic
acid (AQ2A), ethidium bromide (EB), and 9-aminoacridine (9-AA)).

| Compounds | Tₘ (° C.) |
|---|---|
| Control (No DNA Binder) | 19.1 |
| DAPI | 27.8 |
| DNR | 25.4 |
| EB | 21.8 |
| EIPT | 20.5 |
| AMSA | 19.5 |
| AQ2A | 19.4 |
| 9-AA | 19.3 |

### Example 9

### Detection of Triplex DNA Binders

Capture strands of synthetic oligonucleotides are patterned on a glass chip using a microarrayer. The DNA chip is then exposed to a solution containing three components: 1) complementary DNA (no nanoparticles), 2) DNA functionalized Au nanoparticles (DNA is non-complementary to the arrayed DNA) and 3) DNA binders, Scheme 4. The complementary solution-phase DNA, called DNA-3 in Scheme 2, hybridizes to the DNA on the chip surface and forms a duplex. The Au nanoparticle-bound DNA has the correct sequence to form a triplex with the surface bound duplex through sequence-specific Hoogsteen, or reverse Hoogsteen, base pairing. The triplex structure is relatively unstable at room temperature and will only form in the presence of triplex DNA binding molecules. The presence of the triplex DNA binding molecules increase the stability of the triplex, immobilizing the Au nanoparticles on the chip surface. The chip was then exposed to silver enhancement solution for further signal enhancement and then imaged using a Verigene ID scanner.

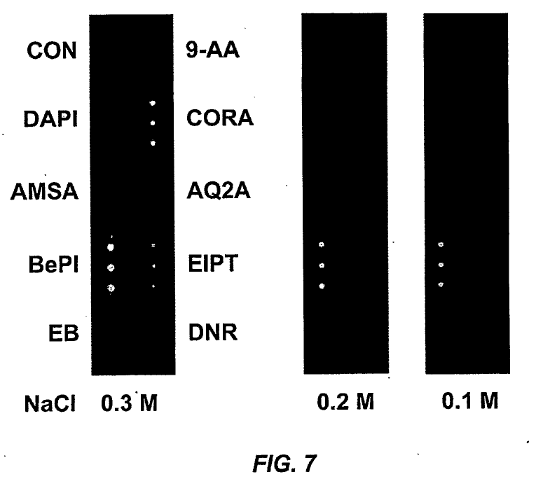

The assay was performed in the presence of two triplex binders, coralyn (CORA) and benzo[e]pyridoindole (BePI), seven duplex DNA binders, 4′,6-diamidino-2-phenylindole (DAPI), ellipticine (EIPT), amsacrine (AMSA), daunorubicin (DNR), anthraquinone-2-carboxylic acid (AQ2A), ethidium bromide (EB), and 9-aminoacridine (9-AA))., and one control (no DNA-3). Similar to the DNA duplex, the formation of the DNA triplex is a reversible process which is affected by both temperature and salt concentration. However, compared to the duplex, the stability of the triplex is much more sensitive to salt concentration. Therefore, to determine the relative binding strength of the triplex DNA binders, the assay was performed at varying salt concentrations, FIG. 7. At 0.3 M NaCl, both the strong (BePI) and weaker (CORA) triplex binders show signal. At decreased salt concentrations (0.2 and 0.1 M NaCl), the stability of the triplex is decreased and signal is only seen due to the stronger triplex binder (BePI). No signal is seen in the presence of the duplex DNA binders indicating that the assay is specific for identifying triplex DNA binders.

These results correlate well with control experiments which measure the Tₘ of solution-phase unmodified DNA in the presence of the two triplex DNA binders and the seven duplex DNA binders, Table 3. A melting transition associated with the triple helix was found only for experiments performed in the presence of BePI (34.8° C.) and CORA (17.1° C.) where BePI is determined to be the stronger binder. This is in agreement with the results determined by the chip-based scanometric method described above.

TABLE 3

| Compounds | Tₘ3-2 (° C.) | Tₘ 2-1 (° C.) |
|---|---|---|
| Control | ND | 58.9 |
| 9-AA | ND | 59.1 |
| AQ2A | ND | 59.1 |
| AMSA | ND | 59.2 |
| EIPT | ND | 59.3 |
| EB | ND | 59.8 |
| CORA | 17.1 | 60.1 |
| BePI | 34.8 | 61.4 |
| DNR | ND | 61.7 |
| DAPI | ND | 66.3 |

From the foregoing it will be appreciated that, although specific embodiments of the invention have been described herein for purposes of illustration, various modifications may be made without deviating from the spirit and scope of the invention.

1. A method for identifying a polynucleotide complex binding compound that stabilizes or destabilizes a duplex polynucleotide complex comprising the steps of
a) contacting a test compound with (i) a first functionalized nanoparticle having a first oligonucleotide attached thereto and (ii) a second functionalized particle having a second oligonucleotide attached thereto, under conditions that permit hybridization between said first oligonucleotide and said second oligonucleotide to form a duplex polynucleotide complex, and
b) identifying the test compound as a duplex polynucleotide complex binding compound that stabilizes or destabilizes said duplex polynucleotide complex when melting temperature of said duplex polynucleotide complex in the presence of said test compound differs from melting temperature of said duplex polynucleotide complex in the absence of said test compound.

2. The method of claim 1, wherein said first oligonucleotide attached to said first nanoparticle and said second oligonucleotide attached to said second nanoparticle are contacted with a free oligonucleotide under conditions that permit formation of a triplex polynucleotide complex, and said test compound is identified as a triplex polynucleotide complex binding compound that stabilizes or destabilizes said triplex polynucleotide complex when melting temperature of said triplex polynucleotide complex in the presence of said test compound differs from melting temperature of said triplex polynucleotide complex in the absence of said test compound.

3.-4. (canceled)

5. The method of claim 1 wherein said first oligonucleotide or said second oligonucleotide is DNA or a modified polynucleotide.

6. The method of claim 1 wherein said first oligonucleotide and said second oligonucleotide are DNA or modified polynucleotides.

7.-8. (canceled)

9. The method of claim 2 wherein said first oligonucleotide, said second oligonucleotide or said free oligonucleotides is DNA or a modified polynucleotide.

10. The method of claim 2 wherein said first oligonucleotide, said second oligonucleotide and said free oligonucleotide are DNA or modified polynucleotides.

11.-12. (canceled)

13. The method of claim 1 or claim 2 wherein said first nanoparticle and said second nanoparticle are gold nanoparticles.

14.-16. (canceled)

17. A method to determine the relative strength of a test duplex polynucleotide complex binding compound compared to a control duplex polynucleotide complex binding compound, comprising the step of
comparing melting temperature of a duplex polynucleotide complex formed between a first oligonucleotide attached to a first nanoparticle and a second oligonucleotide attached to a second nanoparticle and further including the test compound, to melting temperature of a duplex polynucleotide complex formed between the first oligonucleotide attached to the first nanoparticle and the second oligonucleotide attached to the second nanoparticle and further including the control compound,
wherein (i) a higher melting temperature in the presence of the test compound compared to melting temperature in the presence of the control compound indicates stronger binding of the test compound than binding of the control compound, (ii) a lower melting temperature in the presence of the test compound compared to melting temperature in the presence of the control compound indicates weaker binding of the test compound than binding of the control compound, (iii) and melting temperature in the presence of the control compound essentially equal to melting temperature in the presence of the control compound indicates essentially equal binding of the test compound and the control compound.

18. The method of claim 17 wherein said first oligonucleotide or said second oligonucleotide is DNA or a modified polynucleotide.

19. The method of claim 17 wherein said first oligonucleotide and said second oligonucleotide are DNA or modified polynucleotides.

20.-31. (canceled)

32. A method for identifying a polynucleotide complex binding compound that stabilizes or destabilizes a duplex polynucleotide complex comprising the steps of
a) contacting a test compound with (i) a first oligonucleotide immobilized on a substrate and (ii) a functionalized particle having a second oligonucleotide attached thereto, under conditions that permit hybridization between said first oligonucleotide and said second oligonucleotide to form a duplex polynucleotide complex, and
b) identifying the test compound as a duplex polynucleotide complex binding compound that stabilizes or destabilizes said duplex polynucleotide complex when melting temperature of said duplex polynucleotide complex in the presence of said test compound differs from melting temperature of said duplex polynucleotide complex in the absence of said test compound.

33. The method of claim 32, wherein said first oligonucleotide immobilized on the substrate and said second oligonucleotide attached to said nanoparticle are contacted with a free oligonucleotide under conditions that permit formation of a triplex polynucleotide complex, and said test compound is identified as a triplex polynucleotide complex binding compound that stabilizes or destabilizes said triplex polynucleotide complex when melting temperature of said triplex polynucleotide complex in the presence of said test compound differs from melting temperature of said triplex polynucleotide complex in the absence of said test compound.

34.-35. (canceled)

36. The method of claim 32 wherein said first oligonucleotide or said second oligonucleotide is DNA or a modified polynucleotide.

37. The method of claim 32 wherein said first oligonucleotide and said second oligonucleotide are DNA or modified polynucleotides.

38.-39. (canceled)

40. The method of claim 33 wherein said first oligonucleotide, said second oligonucleotide or said free oligonucleotides is DNA or a modified polynucleotide.

41. The method of claim 33 wherein said first oligonucleotide, said second oligonucleotide and said free oligonucleotide are DNA or modified polynucleotides.

42.-43. (canceled)

44. The method of claim 32 or claim 33 wherein said nanoparticle is a gold nanoparticle.

45.-60. (canceled)
